(19) United States
(12) Patent Application Publication
(10) Pub. No.: US 2026/0355227 A1
(43) Pub. Date: Nov. 12, 2026

(54) METHODS OF TREATING INSULIN RESISTANCE AND ASSOCIATED DISORDERS

(71) Applicant: Gallant Bio Pty Ltd, Carlton (AU)

(72) Inventors: Garron DODD, Carlton (AU); Cait BEDDOWS, Carlton (AU); Feiyue SHI, Carlton (AU)

(21) Appl. No.: 19/331,778

(22) Filed: Sep. 17, 2025

(30) Foreign Application Priority Data
Apr. 29, 2025 (WO) PCT/AU2025/050425

(51) Int. Cl. A61K 31/7048; A61K 31/382; A61K 38/26; A61P 3/04; A61P 3/10

(52) U.S. Cl. CPC A61K 31/7048; A61K 31/382; A61K 38/26; A61P 3/04; A61P 3/10

(57) ABSTRACT

The present invention relates to methods of treating or preventing insulin resistance and associated disorders, in particular the use of CS-GAG inhibitors for treating or preventing insulin resistance and associating disorders, such as obesity and type-2 diabetes.

### CROSS-REFERENCE TO RELATED APPLICATION

This application claims the benefit of priority to International Application No. PCT/AU2025/050425, filed Apr. 29, 2025, which is incorporated herein by reference in its entirety.

### TECHNICAL FIELD

The present invention relates generally to methods of treating or preventing insulin resistance and disorders associated with insulin resistance. In particular, the present invention is directed to use of CS-GAG inhibitors for treating or preventing insulin resistance and associated disorders, including metabolic diseases such as obesity and type-2 diabetes.

### BACKGROUND

Metabolic diseases such as obesity and type-2 diabetes mellitus affect around 650 million people worldwide. The global prevalence of metabolic diseases has tripled since 1975 and if current trends continue without the advent of effective treatments, >51% of the world population will be obese or type-2 diabetic by 2030. A key hallmark of many metabolic diseases, including obesity and type-2 diabetes, is insulin resistance.

Insulin is a peptide hormone that is synthesised and secreted by beta cells of the pancreas. Following secretion into the blood, insulin influences a variety of cells expressed throughout the body, where it plays a critical role in maintaining blood glucose levels within an optimum range. In this context, insulin targets peripheral tissues, including the skeletal muscle and adipose tissue to promote glucose uptake from the circulation, and the liver to represses gluconeogenesis and glycogenolysis. Insulin also signals to the brain, where it plays a vital role in how the brain controls whole-body glucose and energy homeostasis. Insulin resistance occurs when insulin's target tissues in the periphery become insensitive to the action of insulin.

Obesity is characterised by an excess accumulation of adipose tissue, which is highly responsive to insulin and contributes greatly to both glucose and lipid metabolism. In obese individuals, adipose tissue releases higher amounts of non-esterified fatty acids, glycerol, hormones, and pro-inflammatory cytokines, which are involved in the development of insulin resistance. Initially, beta cells compensate for insulin resistance by secreting more insulin, but over time beta cells cannot keep up with the body's demand for insulin and blood sugar levels become elevated. Thus, in addition to its own health complications, obesity-associated insulin resistance is also a major risk factor for type-2 diabetes.

Type-2 diabetes can be a difficult disease to manage because it requires long-term maintenance of blood glucose levels. Current antidiabetic drugs do not control blood sugar levels well enough to completely prevent the occurrence of high and low blood sugar levels, which can cause long-term complications such as retinopathy, renopathy, neuropathy and peripheral vascular disease. Many treatments currently available on the market for type-2 diabetes are only partially successful as they target beta cell function or a decrease in insulin resistance and reduce in efficacy as the disease progressively worsens, meaning additional or combination therapies are required over time. People with type-2 diabetes mellitus are also at increased risk of developing other conditions, such as obesity, hypertension, stroke, heart disease and hyperlipidemia.

In addition to metabolic diseases such as obesity and type-2 diabetes, insulin resistance is associated with a host of other serious health problems, including metabolic syndrome, hypertension, dyslipidemia, hyperglycemia, atherosclerosis, non-alcoholic fatty liver disease (NAFLD), polycystic ovary syndrome (PCOS) and coagulopathy.

Glucagon-like peptide 1 receptor (GLP-1R) agonists and analogues thereof, such as liraglutide, stimulate insulin secretion to improve glucose homeostasis in obese and diabetic patients by activating GLP-1 receptors in the arcuate nucleus of the hypothalamus (ARC). However, functional GLP-1 receptor signalling becomes impaired in obese patients and GLP-1 receptor agonists such as liraglutide are less effective in reducing weight and HbA1c level, an index of long-term glycaemic control, for patients with higher BMI levels. Reduced GLP-1-induced insulin release from pancreatic β-cells has also been observed in obese patients with impaired glucose tolerance.

While there are a number of GLP-1R agonists available on the market, they tend to be less effective in patients with a higher BMI. For example, following long-term administration of liraglutide, 45.9% of overweight patients (BMI<30 kg/m²) lost more than 10% of their body weight, as compared with 29.9% of patients with morbid obesity (BMI>40 kg/m²) (Pi-Sunyer, et al., 2015). Clinical efficacy of GLP-1 receptor agonists on improving glycaemic control also appears to wane over time (Davies et al., 2015). Furthermore, I.C.V. administration of GLP-1 impedes lipid uptake into white adipose tissue through the sympathetic nervous system in lean mice rather than in obese mice. Previous attempts to resolve limitations of single GLP-1R agonists have had limited success. For example, single liraglutide treatment has involved simultaneously administering insulin degludec and liraglutide, which can lead to weight gain.

Accordingly, there is a need for improved or alternative methods for treating or preventing insulin resistance and associated disorders, including metabolic diseases such as obesity and type-2 diabetes.

### SUMMARY

The present invention is predicated, at least in part, on the discovery that neurofibrosis develops around metabolically relevant neurons in the arcuate nucleus of the hypothalamus (ARC) during the development of central insulin resistance and metabolic dysfunction, and that CS-GAG inhibitors may reduce or prevent neurofibrosis in the ARC, thereby treating or preventing insulin resistance and associated disorders.

Thus, in one aspect, the present invention provides a method for treating or preventing insulin resistance or an associated disorder in a subject comprising administering an effective amount of a CS-GAG inhibitor, or pharmaceutically acceptable salt, solvate or hydrate thereof, to the subject, wherein the CS-GAG inhibitor is not a 4-epimerase inhibitor.

In another aspect, the present invention provides use of a CS-GAG inhibitor, or pharmaceutically acceptable salt, solvate or hydrate thereof, in the manufacture of a medicament for treating or preventing insulin resistance or an associated disorder in a subject, wherein the CS-GAG inhibitor is not a 4-epimerase inhibitor.

In another aspect, the present invention provides a CS-GAG inhibitor, or pharmaceutically acceptable salt, solvate or hydrate thereof, for use in treating or preventing insulin resistance or an associated disorder in a subject, wherein the CS-GAG inhibitor is not a 4-epimerase inhibitor.

In a further aspect, the present invention provides a pharmaceutical combination comprising (i) a CS-GAG inhibitor, or pharmaceutically acceptable salt, solvate or hydrate thereof, and (ii) a GLP-1 receptor (GLP-1R) agonist, or pharmaceutically acceptable salt, solvate or hydrate thereof, wherein the CS-GAG inhibitor is not a 4-epimerase inhibitor.

In another aspect, the present invention provides a method for treating or preventing insulin resistance or an associated disorder, supressing appetite and/or promoting weight loss in a subject, the method comprising administering to the subject an effective amount of a CS-GAG inhibitor, or pharmaceutically acceptable salt, solvate or hydrate thereof, in combination with a GLP-1R agonist, or pharmaceutically acceptable salt, solvate or hydrate thereof, wherein the CS-GAG inhibitor is not a 4-epimerase inhibitor.

In another aspect, the present invention provides use of a CS-GAG inhibitor, or pharmaceutically acceptable salt, solvate or hydrate thereof, and a GLP-1R agonist, or pharmaceutically acceptable salt, solvate or hydrate thereof, in the manufacture of one or more medicaments for treating or preventing insulin resistance or an associated disorder, supressing appetite and/or promoting weight loss in a subject, wherein the CS-GAG inhibitor is not a 4-epimerase inhibitor.

In another aspect, the present invention provides use of a CS-GAG inhibitor, or pharmaceutically acceptable salt, solvate or hydrate thereof, in the manufacture of a medicament for treating or preventing insulin resistance or an associated disorder, supressing appetite and/or promoting weight loss in a subject, wherein the medicament is formulated for administration with a GLP-1R agonist, or pharmaceutically acceptable salt, solvate or hydrate thereof, wherein the CS-GAG inhibitor is not a 4-epimerase inhibitor.

In another aspect, the present invention provides use of a GLP-1R agonist, or pharmaceutically acceptable salt, solvate or hydrate thereof, in the manufacture of a medicament for treating or preventing insulin resistance or an associated disorder, supressing appetite and/or promoting weight loss in a subject, wherein the medicament is formulated for administration with a CS-GAG inhibitor, or pharmaceutically acceptable salt, solvate or hydrate thereof, wherein the CS-GAG inhibitor is not a 4-epimerase inhibitor.

In another aspect, the present invention provides a combination comprising (i) a CS-GAG inhibitor, or pharmaceutically acceptable salt, solvate or hydrate thereof, and (ii) a GLP-1R agonist, or pharmaceutically acceptable salt, solvate or hydrate thereof, for use in treating or preventing insulin resistance or an associated disorder, supressing appetite and/or promoting weight loss in a subject, wherein the CS-GAG inhibitor is not a 4-epimerase inhibitor.

In embodiments, the CS-GAG inhibitor is not a compound disclosed in WO2024086895, which is incorporated by reference herein. In embodiments, the CS-GAG inhibitor is not fluorosamine.

### BRIEF DESCRIPTION OF THE DRAWINGS

Embodiments of the invention will now be described with reference to the following Figures, which are intended to be exemplary only, and in which:

FIGS. 1 and 2: CS-GAG enzyme knock-out studies show CS-GAG enzyme deficient mice displayed improved whole-body metabolism and glycaemic control in comparison to WT mice. 8 week old C57BL/6J mice received bilateral intraARC injections of an AAV-shRNA targeting CSPG enzymes of interest (FIG. 1a) or a scrambled and placed on a high-fat, high-sugar diet. Body weight (FIG. 1b), appetite and food intake (FIG. 1c-j), glucose tolerance (FIGS. 2a and b) and insulin tolerance (FIGS. 2c and d) were assessed. Results are mean±SEM; significance determined using t-tests, one-way and two-way ANOVA.

FIG. 3: Odiparcil and 4-MUX inhibits in vitro CSPG deposition. Mean fluorescence intensity of WFA staining of CT2A cells treated with increasing doses of Odiparcil and 4-MUX (0, 1, 5, 10, 25 and 50 μm) for 36 hours. Cells were incubated with Odiparcil (FIG. 3A) or 4-MUX (FIG. 3B) in standard DMEM culture medium. Fluorescence intensity values (mean±SEM) were calculated from 3-4 independent wells per treatment group and corrected for DAPI. The Fluorescence intensity values of each treatment group are expressed as a percentage of the vehicle control group (0 μM) was used as the reference for comparisons. Statistical significance was assessed using one-way ANOVA followed by Games-Howell multiple comparisons test against the vehicle control. Significance is denoted as: *p<0.05, **p<0.01, ****p<0.00001.

### DEFINITIONS

Unless defined otherwise, all technical and scientific terms used herein have the same meanings as commonly understood by one of ordinary skill in the art to which this invention belongs.

As used herein, the terms “composition” and “formulation” have been used interchangeably and have the same meaning.

Unless otherwise specified, the indefinite articles “a”, “an” and “the” as used herein, include plural aspects. Thus, for example, reference to “an agent” includes a single agent, as well as two or more agents; reference to “the composition” or “formulation” includes a single composition or formulation, as well as two or more compositions or formulations; and so forth.

As used herein, the term “about” means±10% of the recited value.

Throughout this specification and the claims that follow the word “comprise”, and variations such as “comprises” and “comprising”, will be understood to imply the inclusion of a stated integer or step or group of integers or steps but not the exclusion of any other integer or step or group of integers or steps.

The term “consisting of” means “consisting only of”, that is, including and limited to the integer or step or group of integers or steps, and excluding any other integer or step or group of integers or steps.

The term “consisting essentially of” means the inclusion of the stated integer or step or group of integers or steps, but other integer or step or group of integers or steps that do not materially alter or contribute to the working of the invention may also be included.

The reference to any prior art in this specification is not, and should not be taken as an acknowledgment or any form of suggestion that that prior art forms part of the common general knowledge.

Other definitions are provided throughout the specification.

### DETAILED DESCRIPTION

The present invention relates to methods of treating or preventing insulin resistance and associated disorders, such as type-2 diabetes and obesity. In particular, the present inventors have identified neurofibrosis in the arcuate nucleus of the hypothalamus (ARC) as a novel disease mechanism underlying central insulin resistance and the development of metabolic disease, and that administration of CS-GAG inhibitors may reduce or prevent neurofibrosis in the ARC. The inventors previously identified 4-epimerase may reduce or present neurofibrosis in the ARC. 4-Epimerase (also known as UDP-galactose 4-epimerase) is an enzyme essential for creating the nucleotide sugar substrate UDP-N-acetylgalactosamine required for the assembly and elongation of CS-GAG chain on CSPGs. Surprisingly, the inventors have now identified that enzymes involved in catalyzing the addition of the sugar substrates either directly to the CSPG core protein or to the growing CS-GAG chain also represents a novel treatment for insulin resistance and associated disorders as displayed by improved whole-body metabolism and glycaemic control in mouse knock out studies described herein. Thus, the present invention is therefore directed to the use of CS-GAG inhibitors for treating or preventing insulin resistance and associated disorders, such as type-2 diabetes and obesity, wherein the CS-GAG is not a 4-epimerase inhibitor. The terms “type-2 diabetes mellitus”, “type-2 diabetes” and “T2D” are used interchangeable herein and have the same meaning.

Excessive deposition and remodelling of extracellular matrix (ECM) promotes fibrosis and is an established disease mechanism underpinning insulin resistance within muscle, adipose and liver tissue. However, both insulin resistance and fibrosis have been traditionally viewed as peripheral tissue-centric phenomena and the incidence and relevance of the ECM in the brain to the development of metabolic diseases has not previously been explored. A distinct species of ECM, termed CSPG-ECM, has recently been described in the ARC of humans and mice (Alonge, et al., 2020; Mirzadeh, et al., 2019), which is composed of specialised perisynaptic aggregates of hyaluronic acid, chondroitin sulfate proteoglycans (CSPG) and chondroitin sulfate-glycosaminoglycans (CS-GAG) side chains. The present inventors have identified that the CSPG-ECM within the ARC is a unique multicellular agglomeration concentrated proximally to the median eminence (ME), providing an extracellular juncture between the neural and peripheral endocrine systems. Thus, the CSPG-ECM provides an interface connecting circulating metabolic hormones entering the ARC with metabolically relevant ARC neurons, such as agouti-related peptide neurons (AgRP) and pro-opiomelanocortin (POMC) neurons.

The present inventors have identified that the development of insulin resistance and associated disorders, such as obesity and type-2 diabetes, is underscored by CSPG-ECM remodelling at both the component and glycosaminoglycan level, representing a previously unidentified characteristic of insulin resistance and associated disorders, a phenomenon termed “neurofibrosis”. Neurofibrosis within the ARC impedes the penetrance of circulating insulin, which can result in neuronal insulin resistance. Remodelling of CSPG components, including changes to chondroitin sulfate-glycosaminoglycans sulfation patterns, may be mediated through elevated CS-0S, CS-4S and CS-2S6S sulfation, which promotes a rigid CSPG-ECM structure that sequesters extracellular diffusion. The CSPG-ECM remodelling underscoring neurofibrosis specifically occurs around AgRP neurons, which are critical regulators of metabolism and are essential for survival. Impairments in ARC insulin signalling may promote the development of obesity and diabetes through enhanced feeding behaviour, attenuated energy expenditure and defective glucose metabolism.

A core feature of neurofibrosis in the ARC is the assembly and elongation of CS-GAG chains on CSPGs. The synthesis of CS-GAG chains involves multiple enzymatic steps. These include:
xylosyltransferase 1 and 2 (XylT1 and XylT2) which catalyze the addition of xylose to the CSPG core protein initiating CS-CAG chain formation;
galactosyltransferase 1 and 2 (GalT-I and GalT-II) and glucuronyl transferase 1 (GlcAT-I) which catalyse the addition of galactose (Gal) and glucuronic acid (GlcA) to the xylose residue to form the tetrasaccharide priming sequence (GlcAβ1-3Galβ1-3Galβ1-4Xyl1-O-CSPG core);
chondroitin sulfate N-acetylgalactosaminyltransferase 1 and 2 (CS-GalNAc 1 and CS-GalNAc 2) which catalyze the addition of N-acetylgalactosamine (GalNAc) to the tetrasaccharide primer and growing CS-GAG chains; and
chondroitin sulfate synthases 1 and 3 (CHSY1 and 3) and chondroitin polymerizing factor 2 (Chpf2) which control the polymerization of CS-GAG chains.

A further core feature of neurofibrosis is the enhanced abundance of CSPG core proteins which are covalently bound to the CS-GAG chains in the ARC CSPG-ECM. The CSPG core proteins include versican, phosphocan, neurocan, brevican and aggrecan.

The inventors hypothesized the inhibition of enzymes involved in CS-GAG chain assembly and elongation, including CSPG protein expression, with a CS-GAG inhibitor may reduce or prevent neurofibrosis in the ARC via attenuation of the CSPG-ECM, representing a novel treatment for insulin resistance and associated disorders. As there is little CSPG-ECM expression in the amygdala, orbitofrontal cortex and ventral striatum, targeting the brain ECM may limit off-target effects on depression and anxiety, which have undermined prior attempts to pharmacologically target the brain to treat metabolic disease.

The inventors tested this hypothesis in WT, CS-GAG enzyme knock out mice (as exemplified by CS-GalNAc 1, CHSY1, and CHSY3) and CSPG knock out mice (as exemplified by aggrecan). The results show the CS-GAG enzyme and aggrecan deficient mice displayed improved whole-body metabolism and glycaemic control in comparison to the WT mice. The inventors hypothesise that it would be plausible that knock out studies of other CS-GAG enzymes involved in CS-GAG chain assembly and elongation would display the same. As such, the present invention relates to use of CS-GAG inhibitors for the treatment or prevention of insulin resistance or an associated disorder, wherein the CS-GAG inhibitor is not a 4-epimerase inhibitor.

GLP-1 acts as a metabolic supporter of insulin, as it promotes the insulin secretory response and inhibits glucagon secretion for maintaining glucose homeostasis. GLP-1 is also responsible for enhancing cell survival and triggering the proliferation and differentiation of B-cells. The actions of GLP-1R agonists (including co-agonists such as GIP/GLP-1 and glucagon/GIP/GLP-1 agonists) to promote the remission of metabolic diseases such as obesity and type-2 diabetes is dependent upon the action of these compounds within the ARC. The development of neurofibrosis in the ARC impedes the penetrance, bioavailability and/or signalling of various GLP-1 agonists and insulins into the brain. As GLP-1R agonists cross the blood brain barrier through the same mechanism as GLP-1 co-agonists, such as GIP/GLP-1 receptor agonists and triple glucagon/GIP/GLP-1 receptor agonists, it is also highly plausible that CS-GAG inhibitor treatment will also enhance or improve the activity (e.g., penetrance, bioavailability and/or signalling) of GLP-IR agonists and GLP-1 co-agonists. In some embodiments, the activity of the GLP-1R agonist (or co-agonist) is synergistically enhanced when combined with a CS-GAG inhibitor.

As used herein, an “CS-GAG inhibitor” or “inhibitor of CS-GAG” is any compound that inhibits the activity of an enzyme involved in the assembly and/or elongation of CS-GAG chains including CSPG protein expression, wherein the CS-GAG inhibitor is not a 4-epimerase. Inhibition of activity of the enzyme may also include a reduction in the level or amount of the enzyme protein, RNA or DNA in a cell. The compound may be a competitive, non-competitive, orthosteric, allosteric, or partial inhibitor. In a preferred form the compound is a molecule that inhibits the enzyme activity of the enzyme for example by binding the active site, or competing with the enzyme substrate or co-effector or signaling mechanism.

A non-limiting example of how a CS-GAG inhibitor works in this context is the β-D-thioxyloside, Odiparcil. Odiparcil is a substrate of galactosyltransferase I, an enzyme that catalyses the addition of galactose (Gal) to the xylose residue to form the tetrasaccharide priming sequence (GlcAβ1-3Galβ1-3Galβ1-4Xyl1-O-CSPG core). Odiparcil competes with the natural D-xylose substrate acting as a soluble decoy, diverting GAG synthesis from the proteoglycan core. This produces CS-GAG chains that are linked to Odiparcil. These Odiparcil-linked CS-GAG chains are soluble and can be readily excreted from the body, primarily through urine, leading to a net reduction of cellular CS-GAG chains.

The inhibitor, wherein the inhibitor is not a 4-epimerase, may be specific for the CS-GAG enzyme involved in the assembly and/or elongation of CS-GAG chains and only have some low level inhibitory activity against other receptors (for example, a Ki of greater than about 100 μM, preferably 1 mM against other receptors as measured using an assay as described herein). Preferably, the CS-GAG inhibitor is a substance that limits the activity of the CS-GAG enzyme to 10% or less in comparison with control. Control is a solvent, in which the inhibitor is tested, used at the same quantity, however, without the inhibitor. In some embodiments, the inhibitor may be a small molecule chemical compound, an antisense oligonucleotide (ASO) or interfering RNA (e.g. siRNA). The inhibitor may also be a peptide, including an antibody such as a monoclonal antibody.

The term “small molecule” denotes a generally low molecular weight compound and includes organic and inorganic compounds. In general, a small molecule has a well-defined chemical formula with a single molecular weight. Preferably, a small molecule has a molecular weight of less than 3000 daltons. More preferably, a small molecule has a molecular weight of less than 2000 daltons. In some embodiments of this invention, the small molecule has a molecular weight of less than 1000 daltons. Some non-limiting examples of small molecules include lipids such as fatty acids; saccharides (mono, di or poly); xenobiotics; organometallic compounds and natural products.

The inhibitor of CS-GAG may exhibit a Ki value of less than 1 mM, preferably less than 100 μM, more preferably less than 10 μM, as determined by an assay as described herein. The ability of a compound to inhibit CS-GAG chain assembly and/or elongation be readily determined by one skilled in the art, for example using Western blots for stub chondroitin-4-sulfate attached to the core protein as described by Keough et al., 2016 and Stephenson et al., 2019, the coupled-enzyme system with a spectrophotometric readout as described by Winans and Bertozzi, 2002 or a radiolabel assay using ³⁵SO₄ as described by Esko et al, 1990.

Inhibitors of xylosyltransferases are known in the art and may be useful in the present invention. For example, synthetic xylosides described in Chua et al. Acc. Chem. Res. 2017, 50, 2693-2705 and Esko et al. J Biol Chem, 1991, 266, 6674-6677, and β-D-thioxyloside analogues, such as Odiparcil described in Tallandier et al. PLOS ONE 2020, 15, e0233032.

In embodiments, the CS-GAG inhibitor is selected from the group consisting of amphotericin B, celastrol, 2-Az-Xyl, UDP-4-azido-4-deoxyxylose, 4,4-difluoro-GlcNAc, Odiparcil, β-D-xylosides, ponicidin, or a pharmaceutically acceptable salt, solvate or hydrate thereof. In one embodiment, the β-D-xyloside is not xyloside Ac-bXyl-Teg.

In embodiments, the CS-GAG inhibitor is amphotericin B, or a pharmaceutically acceptable salt, solvate or hydrate thereof. In embodiments, the CS-GAG inhibitor is celastrol, or a pharmaceutically acceptable salt, solvate or hydrate thereof. In embodiments, the CS-GAG inhibitor is 2-Az-Xyl, or a pharmaceutically acceptable salt, solvate or hydrate thereof. In embodiments, the CS-GAG inhibitor is UDP-4-azido-4-deoxyxylose, or a pharmaceutically acceptable salt, solvate or hydrate thereof. In embodiments, the CS-GAG inhibitor is 4,4-difluoro-GlcNAc, or a pharmaceutically acceptable salt, solvate or hydrate thereof. In embodiments, the CS-GAG inhibitor is Odiparcil, or a pharmaceutically acceptable salt, solvate or hydrate thereof. In embodiments, the CS-GAG inhibitor is a β-D-xyloside, or a pharmaceutically acceptable salt, solvate or hydrate thereof, preferably not xyloside Ac-bXyl-Teg. In embodiments, the CS-GAG inhibitor is ponicidin, or a pharmaceutically acceptable salt, solvate or hydrate thereof.

In embodiments, the CS-GAG inhibitor is selected from the group consisting of:

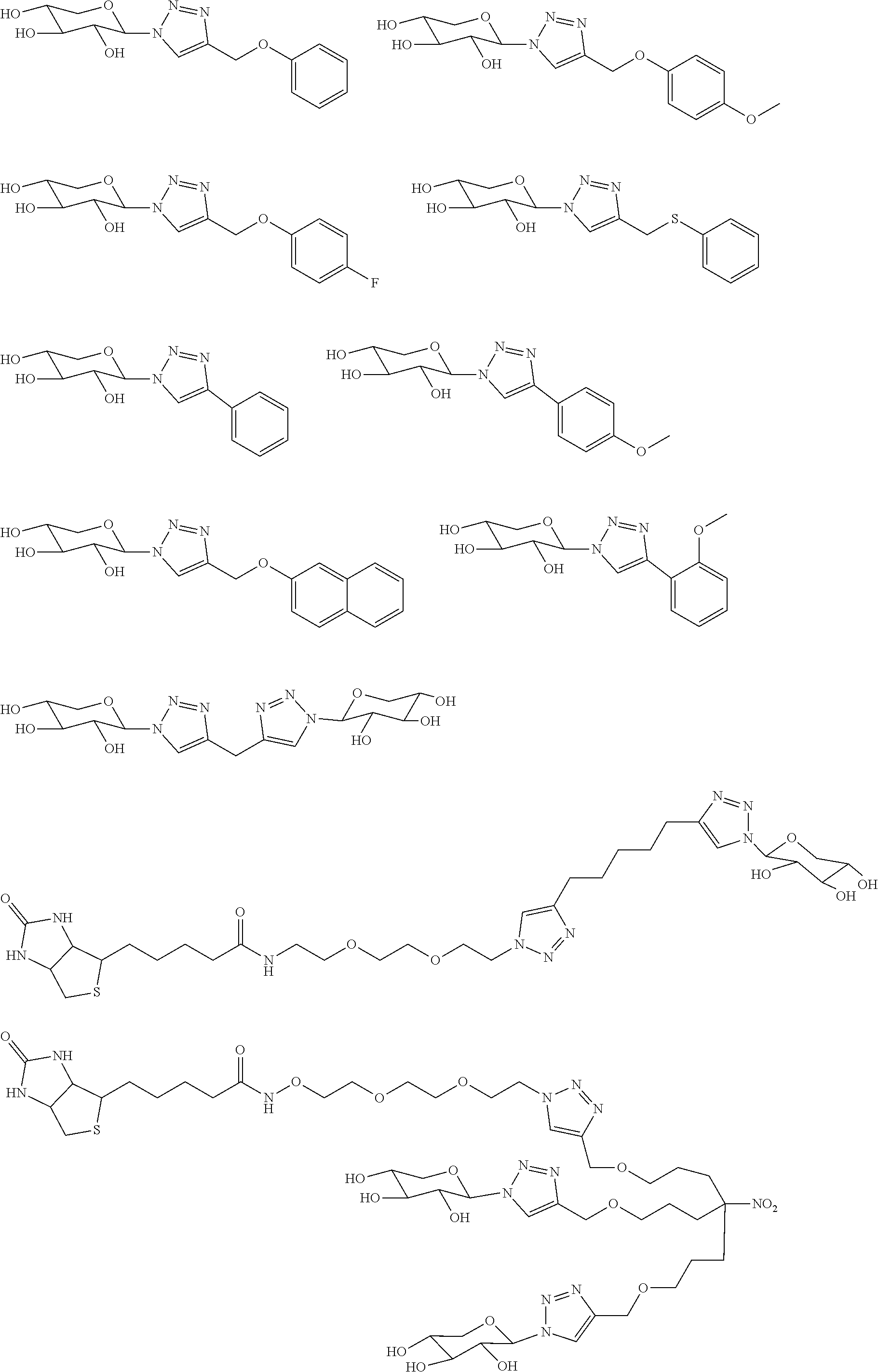

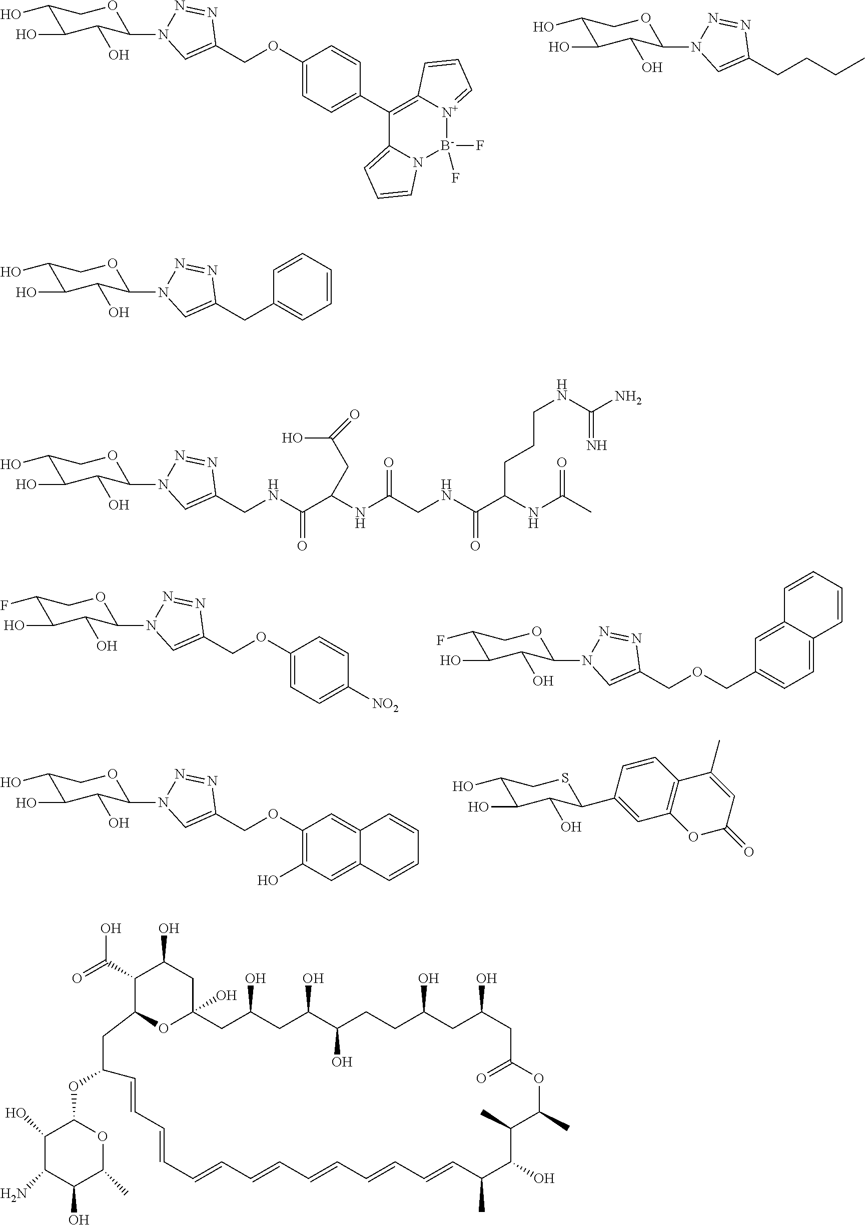

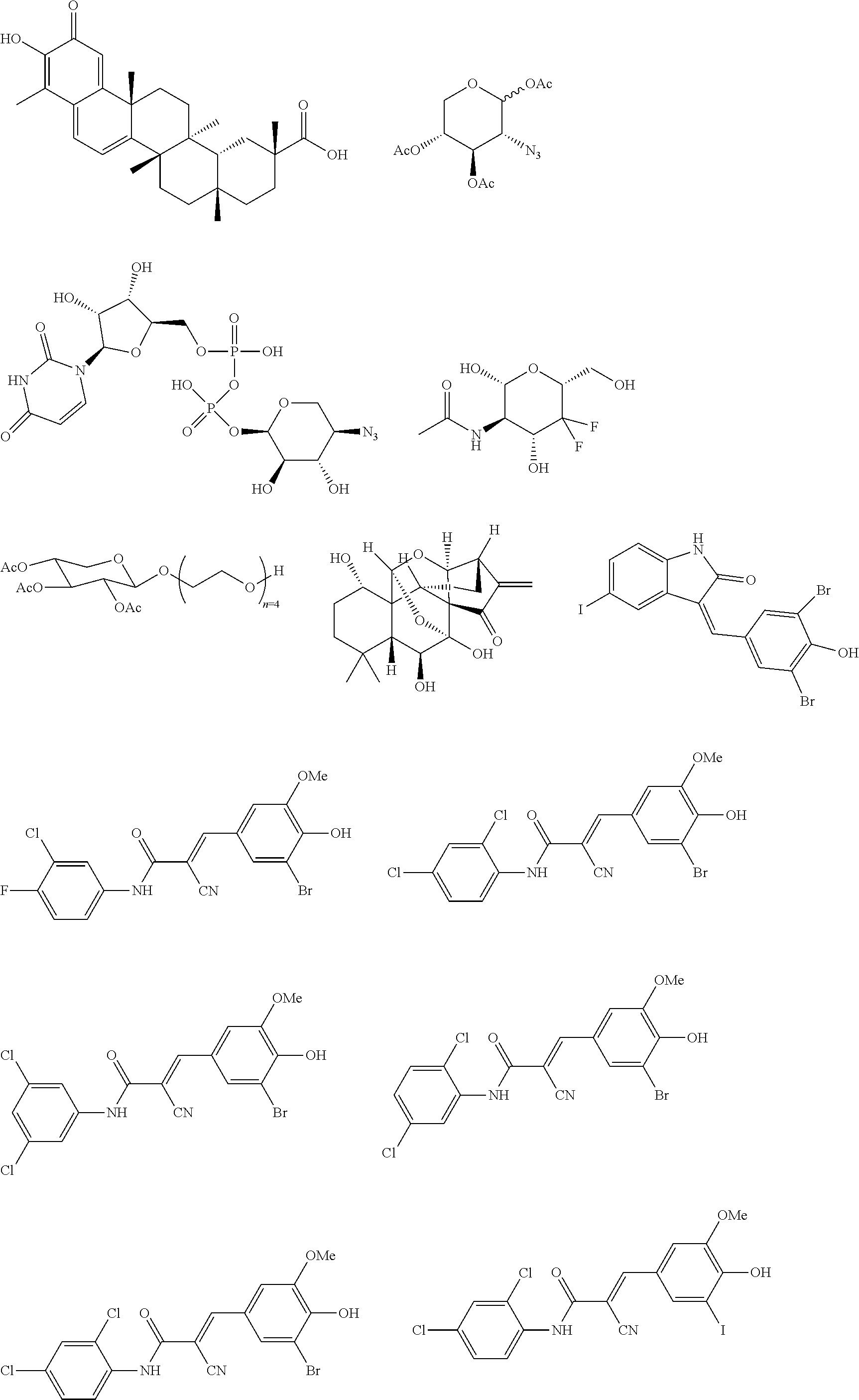

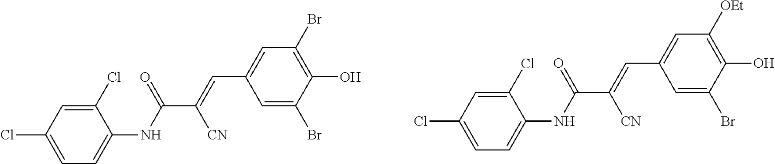 [SMILES: N#C/C(=C\c1cc(Br)c(O)c(Br)c1)C(=O)Nc1ccc(Cl)cc1Cl ; CCOc1cc(/C=C(\C#N)C(=O)Nc2ccc(Cl)cc2Cl)cc(Br)c1O]

a pharmaceutically acceptable salt, solvate or hydrate thereof, and
combinations thereof.

In some embodiments, the CS-GAG inhibitor is not

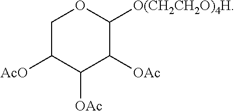

In some embodiments, the present invention relates to the use of a CS-GAG inhibitor, or a pharmaceutically acceptable salt, solvate or hydrate thereof, for the treatment or prevention of insulin resistance or an associated disorder, including metabolic disease associates with insulin resistance, such as obesity and type-2 diabetes mellitus.

In embodiments, the CS-GAG inhibitor is not a 4-epimerase inhibitor.

In one preferred embodiment, the CS-GAG inhibitor is selected from the group consisting of:
xylosyltransferase 1 inhibitor,
xylosyltransferase 2 inhibitor,
galactosyltransferase 1 inhibitor,
galactosyltransferase 2 inhibitor,
glucuronyltransferase 1 inhibitor
chondroitin sulfate N-acetylgalactosaminyltransferase 1 inhibitor,
chondroitin sulfate N-acetylgalactosaminyltransferase 2 inhibitor,
chondroitin sulfate synthase 1 inhibitor,
chondroitin sulfate synthase 2 inhibitor,
chondroitin sulfate synthase 3 inhibitor,
chondroitin polymerizing factor 2 inhibitor,
glycosaminoglycan xylosylkinase (FAM20B) inhibitor,
2-phosphoxylose phosphatase 1 (PXYLP1) inhibitor,
chondroitin 4-O-sulfotransferase 1 inhibitor,
carbohydrate sulfotransferase 15 (GalNac4S-6ST) inhibitor,
carbohydrate sulfotransferase 8 (CHST8) inhibitor,
chondroitin-6-O-sulfotransferase (CS6ST1-2) inhibitor,
an inhibitor of versican expression or synthesis,
an inhibitor of phosphocan expression or synthesis,
an inhibitor of neurocan expression or synthesis,
an inhibitor of brevican expression or synthesis,
an inhibitor of aggrecan expression or synthesis,
a pharmaceutically acceptable salt, solvate or hydrate thereof, and
combinations thereof.

More preferably, the CS-GAG inhibitor is selected from the group consisting of:
galactosyltransferase 1 inhibitor,
chondroitin sulfate N-acetylgalactosaminyltransferase 1 inhibitor,
chondroitin sulfate synthase 1 inhibitor,
chondroitin sulfate synthase 3 inhibitor,
an inhibitor of aggrecan expression or synthesis,
a pharmaceutically acceptable salt, solvate or hydrate thereof, and
combinations thereof.

In some embodiments, the CS-GAG inhibitor is selected from the group consisting of:
chondroitin sulfate N-acetylgalactosaminyltransferase 1 inhibitor,
chondroitin sulfate synthase 1 inhibitor,
chondroitin sulfate synthase 3 inhibitor,
an inhibitor of aggrecan expression or synthesis,
a pharmaceutically acceptable salt, solvate or hydrate thereof, and
combinations thereof.

In some embodiments, the CS-GAG inhibitor is a β-D-xyloside. In some embodiments, the β-D-xyloside is selected from 4-methylumbelliferyl-β-D xylopyranoside (4-MUX), 4-nitrophenyl-β-D xylopyranoside benzyl β-D xylopyranoside a pharmaceutically acceptable salt, solvate or hydrate thereof, and combinations thereof. In some embodiments, the CS-GAG inhibitor is 4-methylumbelliferyl-β-D xylopyranoside or a pharmaceutically acceptable salt, solvate or hydrate thereof

In some embodiments, the CS-GAG inhibitor is selected from the group consisting of:

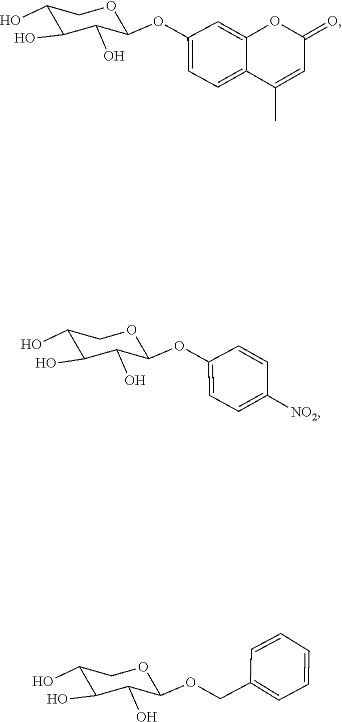 [SMILES: Cc1cc(=O)oc2cc(OC3OCC(O)C(O)C3O)ccc12 ; O=[N+]([O-])c1ccc(OC2OCC(O)C(O)C2O)cc1 ; OC1COC(OCc2ccccc2)C(O)C1O]

a pharmaceutically acceptable salt, solvate or hydrate thereof, and combinations thereof.

In some embodiments, the CS-GAG inhibitor is

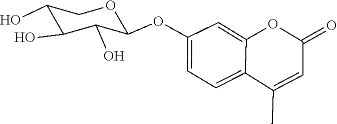 [SMILES: Cc1cc(=O)oc2cc(OC3OCC(O)C(O)C3O)ccc12]

or a pharmaceutically acceptable salt, solvate or hydrate thereof.

In some embodiments, the CS-GAG inhibitor is a β-D-thioxyloside. In some embodiments, the β-D-thioxyloside is selected from Odiparcil, Iliparcil, Beciparil, a pharmaceutically acceptable salt, solvate or hydrate thereof, and combinations thereof Naroparcil. In some embodiments, the β-D-thioxyloside is Odiparcil or a pharmaceutically acceptable salt, solvate or hydrate thereof.

In some embodiments, the CS-GAG inhibitor is selected from the group consisting of:

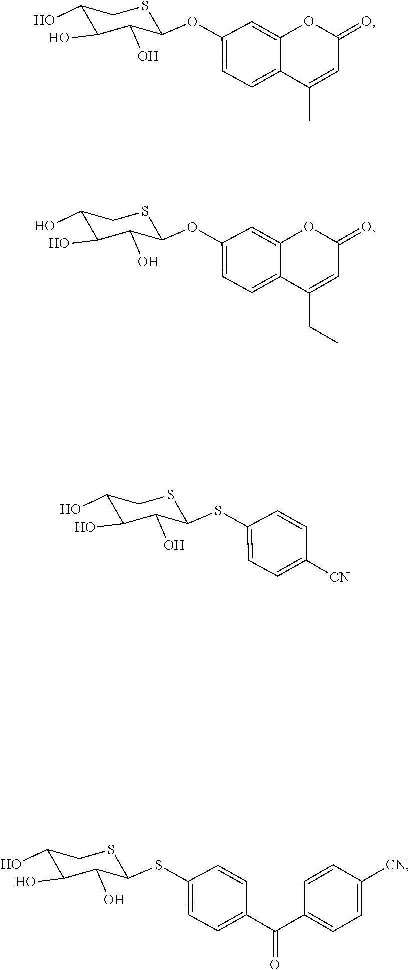 [SMILES: Cc1cc(=O)oc2cc(OC3SCC(O)C(O)C3O)ccc12 ; CCc1cc(=O)oc2cc(OC3SCC(O)C(O)C3O)ccc12 ; N#Cc1ccc(SC2SCC(O)C(O)C2O)cc1 ; N#Cc1ccc(C(=O)c2ccc(SC3SCC(O)C(O)C3O)cc2)cc1]

a pharmaceutically acceptable salt, solvate or hydrate thereof, and combinations thereof.

In some embodiments, the CS-GAG inhibitor is selected from

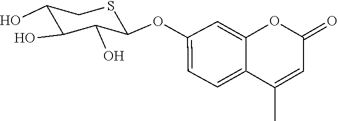 [SMILES: Cc1cc(=O)oc2cc(OC3SCC(O)C(O)C3O)ccc12]

or a pharmaceutically acceptable salt, solvate or hydrate thereof.

In some embodiments, the CS-GAG inhibitor is selected from the group consisting of:
β-D-xyloside,
β-D-thioxyloside,
a pharmaceutically acceptable salt, solvate or hydrate thereof, and
combinations thereof.

In some embodiments, the CG-GAG inhibitor is selected from the group consisting of:
Odiparcil,
4-methylumbelliferyl-β-D xylopyranoside,
a pharmaceutically acceptable salt, solvate or hydrate thereof, and
combinations thereof.

In some embodiments, the CS-GAG inhibitor is selected from the group consisting of:

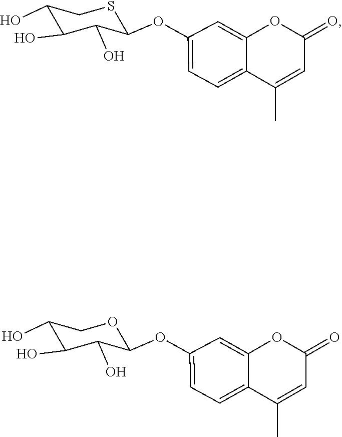 [SMILES: Cc1cc(=O)oc2cc(OC3SCC(O)C(O)C3O)ccc12 ; Cc1cc(=O)oc2cc(OC3OCC(O)C(O)C3O)ccc12]

and, a pharmaceutically acceptable salt, solvate or hydrate thereof, and combinations thereof

In one preferred embodiment, when the CS-GAG inhibitor is a carbohydrate sulfotransferase 15 (GalNac4S-6ST) inhibitor, the CS-GAG inhibitor is

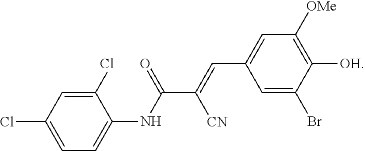 [SMILES: COc1cc(/C=C(\C#N)C(=O)Nc2ccc(Cl)cc2Cl)cc(Br)c1O]

It will be understood that the present invention is not intended to be limited to a particular CS-GAG inhibitor. In view of the mechanisms underlying the present invention as discovered by the present inventors, it is contemplated that any compound that inhibits the activity of an enzyme involved in the assembly and/or elongation of CS-GAG chains may be suitable for use in the present invention. Preferably, the CS-GAG inhibitors are pharmaceutically acceptable compounds.

A skilled person would appreciate the invention also extends to any compound capable of:
degrading the CSPG-ECM,
decreasing the excitability of AgRP,
neutralizing the negative charge of the CSPG chains,
modifications to the sulfation pattern of the CSPG-ECM
and combinations thereof.

Compounds of the invention may be selected from the group consisting of a small molecule, an antibody, a protein, a peptide, and antisense RNA, or an interfering RNA.

It is to be understood that, in accordance with the present invention, the CS-GAG inhibitors may be provided as pharmaceutically acceptable salts, hydrates or solvates. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. The term “solvate” includes a molecular complex comprising a CS-GAG inhibitor and one or more pharmaceutically acceptable solvent molecules, for example, ethanol. The term “hydrate” is employed when the solvent is water. It is also contemplated that CS-GAG inhibitors may be suitable for use in the treatment or prevention of insulin resistance and associated disorders in animals. Thus, term “pharmaceutically acceptable salts” is also intended to include veterinarilly acceptable solvates and hydrates, and veterinarilly acceptable addition salts of the CS-GAG inhibitors.

In some embodiments, pharmaceutically acceptable salts may include acid addition salts and base addition salts. A pharmaceutically acceptable salt involves the inclusion of another molecule such as a chloride ion, an acetate ion, a sulfate ion or other counter ion, in the parent compound. The counter ion may be any organic or inorganic moiety that stabilizes the charge on the parent compound. Furthermore, a pharmaceutically acceptable salt may have more than one charged atom in its structure. When multiple charged atoms are present in the parent compound, its pharmaceutically acceptable salts will have multiple counter ions and these can be several instances of the same counter ion or different counter ions. Hence, a pharmaceutically acceptable salt can have one or more charged atoms in the parent compound and/or one or more counter ions.

Pharmaceutically acceptable acid addition salt refers to those salts which retain the biological effectiveness and properties of the free bases, which are not biologically or otherwise undesirable. Acid addition salts suitable for use in the present invention may be formed from the CS-GAG inhibitor, and a pharmaceutically acceptable inorganic or organic acid, including but not limited to hydrochloric, hydrobromic, sulfuric, phosphoric, methanesulfonic, toluenesulphonic, benzenesulphonic, acetic, propionic, ascorbic, citric, malonic, fumaric, maleic, lactic, salicylic, sulfamic, or tartaric acids. The counter ion of quaternary amines include chloride, bromide, iodide, sulfate, phosphate, methansulfonate, citrate, acetate, malonate, fumarate, sulfamate, and tartrate.

Pharmaceutically acceptable base addition salt refers to those salts that retain the biological effectiveness and properties of the free acids, which are not biologically or otherwise undesirable. Base addition salts suitable for use in the present invention may be formed from the CS-GAG inhibitor, and a pharmaceutically acceptable inorganic or organic base. Salts derived from inorganic bases include, but are not limited to, sodium, potassium, lithium, ammonium, calcium, magnesium, iron, zinc, copper, manganese and aluminum salts. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, dimethylaminoethanol, diethylaminoethanol, dicyclohexylamine, lysine, histidine, arginine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine and polyamine resins. The preparation of the pharmaceutically acceptable salts described above and other typical pharmaceutically acceptable salts is more fully described by Berge et al., “Pharmaceutical Salts,” J. Pharm. Sci., 1977:66:1-19.

Pharmaceutically acceptable solvate refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol.

In some embodiments, salts of CS-GAG inhibitors may be prepared from the free form of the compound in a separate synthetic step prior to incorporation into a formulation for administration to a subject in accordance with the present invention. In still other embodiments, salts of CS-GAG inhibitors may be prepared in situ during preparation of a formulation for administration. For example, the formulations for administration may further comprise an appropriate acid which, upon contact with the free form of the CS-GAG inhibitor forms a desired pharmaceutical salt in situ for administration.

Furthermore, it will be recognised by a person skilled in the art that CS-GAG inhibitors may be provided in crystalline form, either as the free compound or as a solvate (e.g., a hydrate) and it is intended that both forms are within the scope of the present invention. Methods of solvation are generally known within the art.

The present invention also contemplates the use of pharmaceutically acceptable prodrugs of CS-GAG inhibitors in the treatment or prevention of insulin resistance and associated disorders. For example, the CS-GAG inhibitor could be provided in the form of a prodrug, which may, upon administration to a subject, be capable of providing (directly or indirectly) the desired CS-GAG inhibitor, or an active metabolite or residue thereof. The term “prodrug” is used in its broadest sense and encompasses those derivatives that are converted in vivo to the active agent. Such prodrugs would readily occur to those skilled in the art.

As previously described, the present invention encompasses the use of CS-GAG inhibitor as the free base form or as a pharmaceutically salt or solvate thereof in the treatment of insulin resistance or an associated disorder (e.g., a metabolic disease). Where specific dosages or concentrations of a CS-GAG inhibitor are referred to herein, it is to be understood that the specific dosage or concentration refers to the concentration of or equivalent to the free base of the CS-GAG inhibitor. Accordingly, where a pharmaceutically acceptable salt of a CS-GAG inhibitor is used, a person skilled in the art would readily understand that the concentrations or dosages in respect of the salt, refers to the equivalent concentration or dosage of the free base form of the CS-GAG inhibitor.

In accordance with the present invention, CS-GAG inhibitors or pharmaceutically acceptable salts, solvates or hydrates thereof, may be administered together with one or more pharmaceutically acceptable carriers, diluents, adjuvants and/or excipients. Where a carrier, diluent, adjuvants and/or excipient is used, they must be “pharmaceutically acceptable” in the sense of being compatible with the other ingredients of the composition and not injurious to the subject. Such pharmaceutically acceptable carriers, diluents, adjuvants or excipients will be apparent to those skilled in the art and may depend on the intended mode of administration. For example, the carriers, diluents, adjuvants or excipients may vary depending on the formulation and/or mode of administration. In some embodiments, the CS-GAG inhibitor may be provided in sustained-release formulations.

Pharmaceutical compositions comprising a CS-GAG inhibitor for use in the present invention can be prepared by any method known in the art of pharmacology. In general, such preparatory methods include the steps of bringing the CS-GAG inhibitor into association with one or more carriers, diluents, adjuvants, excipients or other accessory ingredients and then, if necessary and/or desirable, shaping and/or packaging the product into a desired single- or multi-dose unit. In certain embodiments, unit dosage compositions are those containing a daily dose or unit, daily sub-dose, as herein above described, or an appropriate fraction thereof, of the CS-GAG inhibitor. As used herein, a “unit dose” is discrete amount of the pharmaceutical composition comprising a predetermined amount of the active ingredient. The amount of the active ingredient (i.e., the CS-GAG inhibitor) is generally equal to the dosage of the active ingredient that would be administered to a subject and/or a convenient fraction of such a dosage such as, for example, one-half or one-third of such a dosage.

General considerations in the formulation and/or manufacture of pharmaceutical compositions can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Pharmaceutical compositions may be formulated from the active agent (i.e., the CS-GAG inhibitor) for any appropriate route of administration. In certain embodiments, compositions in a form suitable for oral use or intranasal use are preferred.

### Therapeutic Use

In accordance with the present invention, CS-GAG inhibitors, or compositions comprising the same, may be used to treat or prevent insulin resistance and associated disorders, wherein the CS-GAG inhibitor is not a 4-epimerase. In the context of the present invention disorders associated with insulin resistance may include disorders caused, at least in part, by insulin resistance (e.g., type-2 diabetes) as well as disorders that themselves cause, at least in part, or exacerbate insulin resistance (e.g., obesity). Such disorders may include, but are not limited to prediabetes, type-2 diabetes mellitus, obesity, metabolic syndrome, hypertension, dyslipidemia, atherosclerosis, non-alcoholic fatty liver disease (NAFLD), polycystic ovary syndrome (PCOS), coagulopathy, insulin resistance syndrome, hyperglycemia, retinopathy, neuropathy, nephropathy, macrovascular disease or combinations thereof.

In accordance with the present invention, CS-GAG inhibitors may be administered to a subject in need of treatment for insulin resistance or an associated disorder, or they may be administered in a prophylactic sense. In particular, it is clear that the methods of the invention may be used prophylactically as well as for the alleviation of symptoms of insulin resistance or an associated disorder. References herein to “treatment” or the like may therefore include such prophylactic treatment, as well as therapeutic treatment of acute conditions or symptoms. Accordingly, in one or more embodiments, the present invention provides CS-GAG inhibitors for use in the therapeutic treatment of insulin resistance or an associated disorder. In other embodiments, the present invention provides CS-GAG inhibitors for use in the prophylactic treatment of insulin resistance or an associated disorder.

Accordingly, the present invention relates to a method of treating or preventing insulin resistance or an associated disorder in a subject comprising administering an effective amount of CS-GAG inhibitor, or pharmaceutically acceptable salt, solvate or hydrate thereof, to the subject, wherein the CS-GAG inhibitor is not a 4-epimerase inhibitor.

In one embodiment, the present invention relates to a method of treating insulin resistance or an associated disorder in a subject comprising administering an effective amount of CS-GAG inhibitor, or pharmaceutically acceptable salt, solvate or hydrate thereof, to the subject, wherein the CS-GAG inhibitor is not a 4-epimerase inhibitor.

In another embodiment, the present invention relates to a method of preventing insulin resistance or an associated disorder in a subject comprising administering an effective amount of CS-GAG inhibitor, or pharmaceutically acceptable salt, solvate or hydrate thereof, to the subject, wherein the CS-GAG inhibitor is not a 4-epimerase inhibitor.

The present invention also relates to use of a CS-GAG inhibitor, or pharmaceutically acceptable salt, solvate or hydrate thereof, in the manufacture of a medicament for treating or preventing insulin resistance or an associated disorder in a subject, wherein the CS-GAG inhibitor is not a 4-epimerase inhibitor.

In one embodiment, the present invention relates to use of a CS-GAG inhibitor, or pharmaceutically acceptable salt, solvate or hydrate thereof, in the manufacture of a medicament for treating insulin resistance or an associated disorder in a subject, wherein the CS-GAG inhibitor is not a 4-epimerase inhibitor.

In another embodiment, the present invention relates to use of a CS-GAG inhibitor, or pharmaceutically acceptable salt, solvate or hydrate thereof, in the manufacture of a medicament for preventing insulin resistance or an associated disorder in a subject, wherein the CS-GAG inhibitor is not a 4-epimerase inhibitor.

The present invention further relates to a CS-GAG inhibitor, or pharmaceutically acceptable salt, solvate or hydrate thereof, for use in treating or preventing insulin resistance or an associated disorder in a subject, wherein the CS-GAG inhibitor is not a 4-epimerase inhibitor.

In one embodiment, the present invention relates to a CS-GAG inhibitor, or pharmaceutically acceptable salt, solvate or hydrate thereof, for use in treating insulin resistance or an associated disorder in a subject, wherein the CS-GAG inhibitor is not a 4-epimerase inhibitor.

In another embodiment, the present invention relates to a CS-GAG inhibitor, or pharmaceutically acceptable salt, solvate or hydrate thereof, for use in preventing insulin resistance or an associated disorder in a subject, wherein the CS-GAG inhibitor is not a 4-epimerase inhibitor.

The terms “treat”, “treating” or “treatment” with regard to a condition (including a disease or disorder as described herein) refers to alleviating or abrogating the cause and/or the effects of the condition. As used herein, the terms “treat”, “treatment” and “treating” refer to the reduction or amelioration of the progression, severity and/or duration of the condition, reversing one or more symptoms of the condition, or the amelioration of one or more symptoms (e.g., one or more discernible symptoms) of the condition (i.e., “managing” without “curing” the condition), resulting from the administration of one or more therapies (e.g., one or more therapeutic agents such as a CS-GAG inhibitor). In specific embodiments, the terms “treat”, “treatment” and “treating” refer to the amelioration of at least one measurable physical parameter of a condition described herein, such as insulin resistance or an associated disorder. In other embodiments the terms “treat”, “treatment” and “treating” refer to the inhibition of the progression of a condition described herein, either physically by, e.g., stabilization of a discernible symptom or physiologically by, e.g., stabilization of a physical parameter, or both.

The terms “preventing” and “prophylaxis” as used herein refer to administering a medicament beforehand to avert or forestall the appearance of one or more symptoms of a condition. The person of ordinary skill in the medical art recognizes that the term “prevent” is not an absolute term. In the medical art, it is understood to refer to the prophylactic administration of a drug to substantially diminish the likelihood or seriousness of a condition, or symptom of the condition and this is the sense intended in this disclosure. As used in a standard text in the field, the Physician's Desk Reference, the terms “prevent”, “preventing” and “prevention” with regard to a condition refer to averting the cause, effects, symptoms or progression of a condition prior to the condition fully manifesting itself.

In some embodiments, the subject in need of treatment or prevention of insulin resistance or an associated disorder is a mammal. The term “mammal” as used herein includes humans, primates, livestock animals (e.g., horses, cattle, sheep, pigs, donkeys), laboratory test animals (e.g., mice, rats, guinea pigs), companion animals (e.g., dogs, cats) and captive wild animals (e.g., kangaroos, deer, foxes). Preferably, the mammal is a human.

In some embodiments, wherein the subject is a human, the subject may be at risk of developing insulin resistance or an associated disorder. The subject may present with one or more risk factors selected from:
obesity,
body mass index greater than or equal to 25 kg/m², 30 kg/m², 35 kg/m² or 40 kg/m²,
hyperglycemia,
fasting blood glucose concentration of greater than or equal to 125 mg/dl, 135 mg/dL, 140 mg/dL, 145 mg/dL, 150 mg/dL, 155 mg/dL or 160 mg/dL,
a fasting high triglyceride (TG) concentration of between about 150 mg/dL and about 500 mg/dL, about 150 mg/dL and about 200 mg/dL, about 200 mg/dL and about 400 mg/dL, about 400 mg/dL and about 500 mg/dL,
hemoglobin A1c levels greater than or equal to 5.7%, 6.0%, 6.5%, 7.0%, 7.5%, 8.0% or 8.5%
a fasting plasma/serum insulin assay greater than 15 mIU/L, 20 mIU/L, 25 mIU/L,
a LDL-c level greater than 100 mg/dL,
a HDL-c level less than 60 mg/dL, and
combinations thereof.

In some embodiments, the methods and uses of the present disclosure include diagnosing a subject with insulin resistance or an associated disorder. In embodiments, the insulin resistance or associated disorder may be diagnosed by measuring or detecting one or more symptoms in the subject selected from:
body mass index greater than or equal to 25 kg/m², 30 kg/m², 35 kg/m² or 40 kg/m²,
hyperglycemia,
fasting blood glucose concentration of greater than or equal to 125 mg/dL, 135 mg/dL, 140 mg/dL, 145 mg/dL, 150 mg/dL, 155 mg/dL or 160 mg/dL,
a fasting high triglyceride (TG) concentration of between about 150 mg/dL and about 500 mg/dL, about 150 mg/dL and about 200 mg/dL, about 200 mg/dL and about 400 mg/dL, about 400 mg/dL and about 500 mg/dL,
hemoglobin A1c levels greater than or equal to 5.7%, 6.0%, 6.5%, 7.0%, 7.5%, 8.0% or 8.5%,
a fasting plasma/serum insulin assay greater than 15 mIU/L, 20 mIU/L, 25 mIU/L,
a LDL-c level greater than 100 mg/dL,
a HDL-c level less than 60 mg/dL,
a Homeostatic Model Assessment of Insulin Resistance (HOMA-IR) score greater 2.5, and
combinations thereof.

In accordance with the present invention, CS-GAG inhibitors are to be administered to the subject in need thereof in a treatment effective amount. In some embodiments, a treatment effective amount is a therapeutically effective amount or a prophylactically effective amount. The term “therapeutically effective amount” as used herein means an amount of a CS-GAG inhibitor sufficient to treat or alleviate the symptoms associated with insulin resistance or an associated disorder. The therapeutically effective amount of the compound to be administered will be governed by such considerations, and is either, an incremental maximum tolerated dose, or the minimum amount, necessary to ameliorate, cure, or treat the condition or one or more of its symptoms. The term “prophylactically effective amount” refers to an amount effective in preventing or substantially lessening the chances of acquiring a disease or disorder or in reducing the severity of the disease or disorder before it is acquired or reducing the severity of one or more of its symptoms before the symptoms develop. Generally, prophylactic measures may be divided between primary prophylaxis (to prevent the development of a disease or symptom) and secondary prophylaxis (whereby the disease or symptom has already developed and the patient is protected against worsening of this process).

As used herein, the term “effective amount” relates to an amount of a CS-GAG inhibitor which, when administered according to a desired dosing regimen, provides the desired therapeutic activity. For example, an effective amount of a CS-GAG inhibitor may be an amount sufficient to inhibit, slow, interrupt, halt, prevent or arrest insulin resistance.

The terms “administer”, “administering” or “administration” in reference to a compound, composition or formulation disclosed herein means introducing the active agent (i.e., the CS-GAG inhibitor) into the system of the subject in need of treatment. When the active agent is provided in combination with one or more other active agents, “administration” and its variants are each understood to include concurrent and/or sequential introduction of the CS-GAG inhibitor and the other active agents.

In some embodiments, a single dose may be sufficient to treat or prevent insulin resistance and associated disorders, which may be delivered in one or more aliquots to achieve the desired dose. In other embodiments, multiple doses may be required to treat or prevent insulin resistance and associated disorders. Dosing may occur at intervals of minutes, hours, days, weeks, months or years or continuously over any one of these periods.

In addition, the administered amount may be an amount sufficient to treat or alleviate the symptoms associated with the insulin resistance or associated disorder.

In one embodiment, an effective dose may be one that, when administered to a subject having insulin resistance or an associated disorder, reduces insulin resistance in the subject relative to that of the subject prior to treatment by at least about 10%, at least about 20%, at least about 25%, at least about 30%, at least about 40%, at least about 50%, at least about 60%, at least about 70%, at least about 80%, or more than 80%.

In some embodiments, an effective dose may be one that, when administered to a subject having insulin resistance or an associated disorder, reduces insulin resistance in the subject relative to that of the subject prior to treatment from about 10% to about 100%, including any value or range therein, including about 10%, about 20%, about 30%, about 40%, about 50%, about 60%, about 70%, about 80%, about 90%, about 100%.

In another embodiment, an effective dose may be one that, when administered to a subject having or at risk of developing insulin resistance or an associated disorder, provides an insulin resistance level in the subject the same or lower as the level in a reference data set in the form of data representative of one or more individuals who do not have a disorder associated with insulin resistance.

In another embodiment, there is provided a method of treating, or reducing the severity of a symptom of insulin resistance or an associated disorder in a subject, the method comprising administering to the subject a therapeutically effective amount of a CS-GAG inhibitor, or pharmaceutically acceptable salt, solvate or hydrate thereof, wherein the CS-GAG inhibitor is not a 4-epimerase inhibitor, thereby treating or reducing the severity of a symptom of insulin resistance in the subject.

A condition or symptom associated with insulin resistance may be any pathology that arises as a consequence of, preceding, or proceeding from the insulin resistance.

In some embodiments, the symptom of insulin resistance is selected from:
obesity,
body mass index greater than or equal to 25 kg/m², 30 kg/m², 35 kg/m² or 40 kg/m²,
hyperglycemia,
fasting blood glucose concentration of greater than or equal to 125 mg/dl, 135 mg/dL, 140 mg/dL, 145 mg/dL, 150 mg/dL, 155 mg/dL or 160 mg/dL
a fasting high triglyceride (TG) concentration of between about 150 mg/dL and about 500 mg/dL, about 150 mg/dL and about 200 mg/dL, about 200 mg/dL and about 400 mg/dL, about 400 mg/dL and about 500 mg/dL,
hemoglobin A1c levels greater than or equal to 5.7%, 6.0%, 6.5%, 7.0%, 7.5%, 8.0% or 8.5%
a fasting plasma/serum insulin assay greater than 15 mIU/L, 20 mIU/L, 25 mIU/L,
a LDL-c level greater than 100 mg/dL,
a HDL-c level less than 60 mg/dL, and
combinations thereof.

An effective dose may be one that, when administered to the subject reduces the severity of a condition or symptom associated with insulin resistance.

Obesity is classified by body mass index (BMI) into classes I, II and III. Successful treatment may be determined by a reduction in the severity of obesity. A reduction in the severity of obesity refers to a subject presenting with an obese BMI classification (such as Obese class III, Obese class II or Obese class I) prior to treatment, and presenting with a lower BMI classification after treatment. For example, a subject may present as Obese class III prior to treatment and present as Obese class II, Obese class I, Overweight, Healthy weight, or Underweight, after treatment. In another example, a subject may present as Obese class I prior to treatment and present as Overweight, Healthy weight, Underweight, or any class therein, after treatment.

Successful treatment may be determined by a reduction in body mass index. In one embodiment, the body mass index of the subject reduces by at least about 10%, at least about 20%, at least about 25%, at least about 30%, at least about 40%, at least about 50%. In another embodiment, the body mass index of the subject after treatment is less than or equal to 35 kg/m², 30 kg/m², 25 kg/m², or 20 kg/m².

Successful treatment may be determined by a reduction in body weight. In one embodiment, the body weight of the subject reduces by about 1% to 10%, including at least about 1%, at least about 2%, at least about 3%, at least about 4%, at least about 5%, at least about 6%, at least about 7%, at least about 8%, at least about 9%, at least 10%. Preferably, the body weight of the subject reduces by about 1% to 5%, including at least about 1%, at least about 2%, at least about 3%, at least about 4%, at least about 5%, most preferably by at least about 5%.

Hyperglycemia due to insulin resistance is characterized by elevated blood glucose levels and may be classified as severe hyperclycemia, moderate hyperclycemia and mild hyperclycemia. Successful treatment may be determined by a reduction in the severity of hyperclycemia. For example, a subject may have severe hyperclycemia prior to treatment and have moderate or mild hyperclycemia after treatment. In another example, a subject may have moderate hyperclycemia prior to treatment and have mild hyperclycemia after treatment.

Successful treatment may be determined by a reduction in fasting blood glucose concentration. In one embodiment, the fasting blood glucose concentration of the subject reduces by at least about 10%, at least about 20%, at least about 25%, at least about 30%, at least about 40%, at least about 50%. In another embodiment, the fasting blood glucose concentration of the subject after treatment is less than 125 mg/dL, most preferably 70-100 mg/dL.

Successful treatment may be determined by a reduction in fasting high triglyceride (TG) concentration. In one embodiment, the fasting high triglyceride (TG) concentration of the subject reduces by at least about 10%, at least about 20%, at least about 25%, at least about 30%, at least about 40%, at least about 50%. In another embodiment, the fasting high triglyceride (TG) concentration of the subject after treatment is less than 200 mg/dL, preferably less than 150 mg/dL.

Successful treatment may be determined by a reduction in hemoglobin A1c levels. In one embodiment, the hemoglobin A1c levels of the subject reduces by at least about 10%, at least about 20%, at least about 25%, at least about 30%, at least about 40%, at least about 50%. In another embodiment, the hemoglobin A1c levels of the subject after treatment is less than 6.5%, 6.0%, most preferably less than 5.7%.

Successful treatment may be determined by a reduction in fasting plasma/serum insulin concentration. In one embodiment, the fasting plasma/serum insulin concentration of the subject reduces by at least about 10%, at least about 20%, at least about 25%, at least about 30%, at least about 40%, at least about 50%. In another embodiment, the fasting plasma/serum insulin concentration of the subject after treatment is less than 25 mIU/L, 20 mIU/L, preferably 5-15 mIU/L.

Successful treatment may also be determined by assessing a subject's Homeostatic Model Assessment of Insulin Resistance (HOMA-IR) score. In one embodiment, the fasting plasma/serum insulin concentration of the subject reduces by at least about 10%, at least about 20%, at least about 30%, at least about 40%, at least about 50%, at least about 60%, at least about 70%, at least about 80%, at least about 90%, at least about 95%. In another embodiment, the Homeostatic Model Assessment of Insulin Resistance (HOMA-IR) score of the subject after treatment is about 2.0, 1.75, 1.50, 1.25, 1.00, or 0.50.

Successful treatment may also be determined by assessing a subject's C-peptide levels. In one embodiment, the fasting plasma/serum C-peptide concentration of the subject reduces by at least about 10%, at least about 20%, at least about 30%, at least about 40%, at least about 50%, at least about 60%, at least about 70%, at least about 80%, at least about 90%, at least about 95%. In another embodiment, the C-peptide concentration of the subject after treatment is at or below 2.5 ng/ml, 2.0 ng/mL, 1.5 ng/ml, 1.0 ng/ml, or 0.5 ng/mL.

In some embodiments, the CS-GAG inhibitor may be administered, or formulated for administration, orally.

In another embodiment, the CS-GAG inhibitor may be administered, or formulated for administration, intranasally.

The amount of CS-GAG inhibitor administered per dose or the total volume of composition administered will depend on such factors as the nature and severity of the symptoms, the age, weight, and general health of the patient, as well as the mode of administration. It is recognised that relative amounts of excipients, solvents, diluents, salts, thickening agents, sensory agents, buffers, and/or any additional ingredients in a pharmaceutical composition as disclosed herein may also depending upon the identity, size, and/or condition of the subject treated, as well as the mode of administration. For example, in some embodiments, the dosage of CS-GAG inhibitor required to achieve a therapeutically equivalent effect may be greater for dosage form compared to another. The terms “therapeutic equivalence” or “therapeutically equivalent” as used herein refer to different compositions comprising the same active agent that produce the same clinical effect and safety profile and/or are pharmaceutical equivalents to one another.

Formulations comprising the CS-GAG inhibitor may be administered in a single dose or a series of doses. Suitable dosage amounts and dosing regimens can be determined by the attending physician and may depend on the particular condition being treated, the severity of the condition as well as the general age, health and weight of the subject. It will be appreciated that dose ranges as described herein provide guidance for the administration of provided pharmaceutical compositions to an adult. The amount to be administered can be determined by a medical practitioner or person skilled in the art.

In certain embodiments, it is envisaged that a CS-GAG inhibitor may be administered to a subject in need thereof as a substitute or replacement for other traditional medication for the treatment of insulin resistance or an associated disorder. In other embodiments, it is envisaged that a CS-GAG inhibitor be administered to a subject in need thereof as a supplement or adjunct to traditional medication. In still other embodiments, it is envisaged that a CS-GAG inhibitor may be administered to a subject in need thereof in the absence of adjunct therapy. Replacing traditional medication for the treatment of metabolic diseases with a CS-GAG inhibitor may be advantageous, particularly where the traditional medication is associated with one or more adverse effects.

### Combination Therapy

In some embodiments, a CS-GAG inhibitor, or pharmaceutically acceptable salt, solvate or hydrate thereof, may be administered to a subject in need thereof, together with one or more additional therapeutic agents for a discrete period of time, to address specific symptoms of insulin resistance or an associated disorder, wherein the CS-GAG inhibitor is not a 4-epimerase inhibitor.

Increased appetite and/or weight may be associated with insulin resistance or an associated disorder, or they may associated with insulin dysregulation (e.g., as a precursor to or risk factor for insulin resistance or an associated disorder). Thus, the combinations disclosed herein may be particularly suitable for suppressing appetite and/or promoting weight loss associated with insulin dysregulation. In particular, a CS-GAG inhibitor and a GLP-1R agonist may be administered in combination to a subject in need of treatment (e.g., for insulin resistance or an associated disorder, to suppress appetite or promotion weight loss) or they may be administered in a prophylactic sense. Accordingly, in one or more embodiments, the present invention provides a CS-GAG inhibitor in combination with a GLP-1R agonist for use in the therapeutic treatment of insulin resistance or an associated disorder, to suppress appetite and/or to promote weight loss. In other embodiments, the present invention provides a CS-GAG inhibitor in combination with a GLP-1R agonist for use in the prophylactic treatment of insulin resistance or an associated disorder, to suppress appetite and/or to promote weight loss.

A skilled person would appreciate the invention extends to a CS-GAG-GLP-IR agonist conjugate.

Accordingly, in some embodiments, the present invention relates to a method of treating or preventing insulin resistance or an associated disorder in a subject comprising administering to the subject an effective amount of a CS-GAG inhibitor in combination with a GLP-1R agonist, wherein the CS-GAG inhibitor is not a 4-epimerase inhibitor.

The CS-GAG inhibitor and GLP-1R agonist may be administered in a single dosage form, or discrete dosage forms.

A single dosage form may include co-formulation of the CS-GAG inhibitor, or pharmaceutically acceptable salt, solvate or hydrate thereof, and the GLP-1R agonist, or pharmaceutically acceptable salt, solvate or hydrate thereof. The co-formulation may comprise a mixture of the CS-GAG inhibitor, or pharmaceutically acceptable salt, solvate or hydrate thereof, and the GLP-1R agonist, or pharmaceutically acceptable salt, solvate or hydrate thereof. Alternatively, the co-formulation may comprise a complex formed from the CS-GAG inhibitor, GLP-1R agonist, and pharmaceutically acceptable salt(s), solvate(s) or hydrate(s) thereof.

Discrete dosage forms may be administered concurrently and/or sequentially in either order.

In one embodiment, the present invention relates to a method of treating insulin resistance or an associated disorder in a subject comprising administering to the subject an effective amount of a CS-GAG inhibitor in combination with a GLP-1R agonist, wherein the CS-GAG inhibitor is not a 4-epimerase inhibitor.

In another embodiment, the present invention relates to a method of preventing insulin resistance or an associated disorder in a subject comprising administering to the subject an effective amount of a CS-GAG inhibitor in combination with a GLP-1R agonist, wherein the CS-GAG inhibitor is not a 4-epimerase inhibitor.

The present invention also relates to a method of suppressing appetite and/or promoting weight loss in a subject comprising administering to the subject an effective amount of a CS-GAG inhibitor in combination with a GLP-1R agonist, wherein the CS-GAG inhibitor is not a 4-epimerase inhibitor.

The present invention also relates to use of a CS-GAG inhibitor and a GLP-IR agonist in the manufacture of one or more medicaments for treating or preventing insulin resistance or an associated disorder, suppressing appetite and/or promoting weight loss in a subject, wherein the CS-GAG inhibitor is not a 4-epimerase inhibitor.

In one embodiment, the present invention relates to use of a CS-GAG inhibitor and a GLP-1R agonist in the manufacture of one or more medicaments for treating insulin resistance or an associated disorder, suppressing appetite and/or promoting weight loss in a subject, wherein the CS-GAG inhibitor is not a 4-epimerase inhibitor.

In another embodiment, the present invention relates to use of a CS-GAG inhibitor and a GLP-1R agonist in the manufacture of one or more medicaments for preventing insulin resistance or an associated disorder, suppressing appetite and/or promoting weight loss in a subject, wherein the CS-GAG inhibitor is not a 4-epimerase inhibitor.

The present invention also relates to use of a CS-GAG inhibitor in the manufacture of a medicament for treating or preventing insulin resistance or an associated disorder, supressing appetite and/or promoting weight loss in a subject, wherein the medicament is formulated for administration with a GLP-1R agonist, wherein the CS-GAG inhibitor is not a 4-epimerase inhibitor.

In one embodiment, the present invention relates to use of a CS-GAG inhibitor in the manufacture of a medicament for treating insulin resistance or an associated disorder, supressing appetite and/or promoting weight loss in a subject, wherein the medicament is formulated for administration with a GLP-1R agonist, wherein the CS-GAG inhibitor is not a 4-epimerase inhibitor.

In one embodiment, the present invention relates to use of a CS-GAG inhibitor in the manufacture of a medicament for preventing insulin resistance or an associated disorder, supressing appetite and/or promoting weight loss in a subject, wherein the medicament is formulated for administration with a GLP-1R agonist, wherein the CS-GAG inhibitor is not a 4-epimerase inhibitor.

The present invention also relates to use of a GLP-1R agonist in the manufacture of a medicament for treating or preventing insulin resistance or an associated disorder, supressing appetite and/or promoting weight loss in a subject, wherein the medicament is formulated for administration with a CS-GAG inhibitor, wherein the CS-GAG inhibitor is not a 4-epimerase inhibitor.

In one embodiment, the present invention relates to use of a GLP-1R agonist in the manufacture of a medicament for treating insulin resistance or an associated disorder, supressing appetite and/or promoting weight loss in a subject, wherein the medicament is formulated for administration with a CS-GAG inhibitor, wherein the CS-GAG inhibitor is not a 4-epimerase inhibitor.

In one embodiment, the present invention relates to use of a GLP-1R agonist in the manufacture of a medicament for preventing insulin resistance or an associated disorder, supressing appetite and/or promoting weight loss in a subject, wherein the medicament is formulated for administration with a CS-GAG inhibitor, wherein the CS-GAG inhibitor is not a 4-epimerase inhibitor.

The present invention further relates to a combination comprising a CS-GAG inhibitor and a GLP-1R agonist for use in treating or preventing insulin resistance or an associated disorder, suppressing appetite and/or promoting weight loss in a subject, wherein the CS-GAG inhibitor is not a 4-epimerase inhibitor.

The combination includes the CS-GAG inhibitor and GLP-1R agonist as discrete dosage forms that may be administered concurrently and/or sequentially in either order. The combination also includes co-formulations of the CS-GAG inhibitor and GLP-1R agonist in a single dosage form. The co-formulation may comprise a mixture of the CS-GAG inhibitor, or pharmaceutically acceptable salt, solvate or hydrate thereof, and the GLP-1R agonist, or pharmaceutically acceptable salt, solvate or hydrate thereof. Alternatively, the co-formulation may comprise a complex formed from the CS-GAG inhibitor, GLP-1R agonist, and pharmaceutically acceptable salt(s), solvate(s) or hydrate(s) thereof.

In one embodiment, the present invention relates to a combination comprising a CS-GAG inhibitor and a GLP-1R agonist for use in treating insulin resistance or an associated disorder, suppressing appetite and/or promoting weight loss in a subject, wherein the CS-GAG inhibitor is not a 4-epimerase inhibitor.

In one embodiment, the present invention relates to a combination comprising a CS-GAG inhibitor and a GLP-1R agonist for use in preventing insulin resistance or an associated disorder, suppressing appetite and/or promoting weight loss in a subject, wherein the CS-GAG inhibitor is not a 4-epimerase inhibitor.

In some embodiments, the associated disorder is selected from prediabetes, type-2 diabetes mellitus, obesity, metabolic syndrome, hypertension, dyslipidemia, atherosclerosis, non-alcoholic fatty liver disease (NAFLD), polycystic ovary syndrome (PCOS), coagulopathy and obstructive sleep apnoea.

In some embodiments, the associated disorder is a metabolic disease. Preferably, type-2 diabetes mellitus.

In some embodiments, the method is for treating obesity, suppressing appetite and/or promoting weight loss.

In some embodiments, the method disclosed herein promotes weight loss in a subject, reducing the weight of the subject by at least about 5%, 10%, 15%, 20%, 25% over 3-36 months as compared to the subject prior to the treatment.

In some embodiments, the CS-GAG inhibitor is administered orally.

In some embodiments, the GLP-1 agonist is administered by injection or orally.

In some embodiments, the GLP-1 agonist is administered by injection.

In some embodiments, the GLP-1 agonist is administered orally.

The GLP-1 receptor (GLP-1R) agonists (also referred to as a “GLP-1 agonist”) used in the combinations of the present invention may be any GLP-1R agonist, including GLP-1 or an analogue thereof. It is to be understood that such analogues are limited to analogues that maintain the ability (at least in part) to agonise GLP-1R. A number of suitable GLP-1R agonists are currently available on the market, including liraglutide (Saxenda™, Voctoza™), dulaglutide (Trulicity™), exenatide (Bydureon™, Byetta™), semaglutide (Ozempic™) and lixisenatide (Lyxumia™, Adlyxin™), among others. In a particular embodiment, the GLP-1R agonist is liraglutide. However, it is to be understood that the GLP-1R agonist may include both chemical compounds (e.g., small molecules) and biological compounds (e.g., peptides, including recombinant fusion proteins and synthetic peptides), which may or may not be commercially available. Other non-limiting examples of GLP-1R agonists can be found in WO 98/08873, WO 2016/097108, WO 2021/187886, WO 2021/160127, the entire contents of each of which are incorporated herein by cross-reference. Other suitable GLP-1R agonists (including GLP-1 and analogues thereof) and processes for their preparation will be known to those skilled in the art.

The term GLP-1R agonist is intended to encompass GLP-IR co-agonists (also referred to as “GLP-1 co-agonists”), including dual agonists, triple agonists, and the like. Examples of suitable co-agonists may include, but are not limited to, GIP/GLP-1 co-agonists/glucacon/GLP-1 co-agonists, and glucagon/GIP/GLP-1 co-agonists. In an embodiment, the GLP-1 agonist is a GIP/GLP-1 co-agonist. An example of a commercially available GIP/GLP-1 co-agonist that may be suitable for use in the present invention is tirzepatide (Mounjaro™). Other non-limiting examples of GIP/GLP-1 co-agonists are described in WO 2011/119657, WO 2016/111971, WO 2013/164483, WO 2014/192284 and WO 2015/086729, the entire contents of each of which are incorporated herein by cross-reference. In another embodiment, the GLP-1 agonist is a glucacon/GLP-1 co-agonist. Non-limiting examples of GLP-1/glucacon co-agonists are described in WO 2008/101017 and WO 2017/100107, the entire contents of each of which are incorporated herein by cross-reference. In another embodiment, the GLP-1 agonist is a glucagon/GIP/GLP-1 co-agonist. An example of a glucagon/GIP/GLP-1 co-agonist currently in Phase I clinical trials is LY3437943 (Coskun et al., 2022). Other GLP-1 co-agonists are also contemplated by the present invention.

In some embodiments, the GLP-1R agonist is GLP-1 or an analogue thereof. Preferably, the GLP-1R agonist is selected from liraglutide dulaglutide, exenatide, semaglutide and lixisenatide, or any combination thereof. More preferably, the GLP-1R agonist is liraglutide.

In some embodiments, the GLP-1R agonist is a GLP-IR co-agonist. Preferably, the GLP-1R co-agonist is tirzepatide or LY3437943.

It will be understood that the present invention is not intended to be limited to any particular GLP-1R agonists described herein. It is contemplated that any agonists of the GLP-1 receptor may also be suitable for use in the combinations of the present invention. The ability of a compound to agonise GLP-1R may be readily determined by one skilled in the art, for example using GLP-1 and GIP receptor binding assays or receptor internalisation assays.

In still other embodiments, the subject in need thereof may be treated with a CS-GAG inhibitor and one or more additional therapeutic agents (administered sequentially or in combination) for the duration of the treatment period. Such combination therapy may be particularly useful, for example, where an additive or synergistic therapeutic effect is desired. Where the active agents are provided in separate dosage formulations, the active agents may be administered separately or in conjunction. In addition, the administration of one active agent may be prior to, concurrent with, or subsequent to the administration of the other agent.

The phrase “combination therapy” as used herein, is to be understood to refer to administration of an effective amount, using a first amount of, for example, a CS-GAG inhibitor, and a second amount of an additional suitable therapeutic agent, for examples a GLP-1R agonist. An “effective amount” of the second agent will depend on the type of drug used. Suitable dosages are known for approved agents and can be adjusted by a person skilled in the art according to the condition of the subject, the type of condition(s) being treated and the amount of a compound or composition being used. In certain embodiments, the CS-GAG inhibitor and the additional therapeutic agent are each administered in an effective amount (i.e., each in an amount that would be therapeutically effective if administered alone). In other embodiments, the CS-GAG inhibitor and the additional therapeutic agent are each administered in an amount that alone does not provide a therapeutic effect (a sub-therapeutic dose). In yet other embodiments, the CS-GAG inhibitor can be administered in an effective amount, while the additional therapeutic agent is administered in a sub-therapeutic dose. In still other embodiments, the CS-GAG inhibitor can be administered in a sub-therapeutic dose, while the additional therapeutic agent is administered in an effective amount.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a person in need thereof. Co-administration encompasses administration of the CS-GAG inhibitor and one or more additional therapeutic agents in an essentially simultaneous manner, such as in a single pharmaceutical composition, for example, having a fixed ratio of first and second amounts, or as discrete dosage forms. In addition, such co-administration also encompasses use of each compound in a sequential manner in either order. When co-administration involves the separate administration of a first amount of a CS-GAG inhibitor and a second amount of an additional therapeutic agent, they are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life, and kinetic profile.

In one or more embodiments where the CS-GAG inhibitor is administered in combination with an additional therapeutic agent, the additional therapeutic agent may be any therapeutic agent that provides a desired treatment outcome. In particular, the additional therapeutic agent may be selected from known therapeutic agents for the treatment or prevention of insulin resistance or an associated disorders, including one or more symptoms thereof. Such therapeutic agent will be known to those skilled in art. By way of non-limiting example, known therapeutic agents for the treatment of obesity or type-2 diabetes, which may be suitable for use in combination with CS-GAG inhibitors in the present invention.

When the additional therapeutic agent is a GLP-1R agonist, it is to be understood that the active agents in the combination (i.e., the CS-GAG inhibitor and GLP-1R agonist) may be administered at a dose and/or frequency that is more or less than the effective amount for each active agent alone. In particular, where the combination provides a synergistic effect, a lower dose and/or frequency may be equally or more effective than if one of the component active agents are administered alone. In certain embodiments, the CS-GAG inhibitor and the GLP-1R agonist are each administered in an effective amount (i.e., each in an amount that would be therapeutically effective if administered alone). In other embodiments, the CS-GAG inhibitor and the GLP-1R agonist are each administered in an amount that alone does not provide a therapeutic effect (a sub-therapeutic dose). In yet other embodiments, the CS-GAG inhibitor can be administered in an effective amount, while the GLP-1R agonist is administered in a sub-therapeutic dose. In still other embodiments, the CS-GAG inhibitor can be administered in a sub-therapeutic dose, while the GLP-1R agonist is administered in an effective amount.

Where a CS-GAG inhibitor is administered in combination with an additional therapeutic agent, the additional agent may be administered in any “effective amount” which provides the desired therapeutic activity, as described above. Suitable dosage amounts and dosing regimens of the additional therapeutic agent can be determined by the attending physician and may depend on the particular condition being treated, the severity of the condition as well as the general age, health and weight of the subject. It will be appreciated that, unless otherwise specified, dose ranges as described herein provide guidance for the administration of provided pharmaceutical compositions to an adult. The amount to be administered to can be determined by a medical practitioner or person skilled in the art.

The CS-GAG inhibitor and formulations thereof may be contained in a kit. The kit may include, for example, the CS-GAG inhibitor and an additional agent, each packaged or formulated individually, or packaged or formulated in combination. Thus, the CS-GAG inhibitor may be present in a first container, and the kit can optionally include one or more agents in a second container. The container or containers are placed within a package, and the package can optionally include administration or dosage instructions. The kits disclosed herein may comprise the CS-GAG inhibitor in a form suitable for oral or intranasal administration. The kits may optionally comprise instructions describing a method of using the pharmaceutical compositions in one or more of the methods described herein (e.g., for preventing or treating a metabolic disease). The kit may optionally comprise a second pharmaceutical composition comprising one or more additional agents described herein for co-therapy use, a pharmaceutically acceptable carriers, diluents, adjuvants and/or excipients. The pharmaceutical composition comprising the CS-GAG inhibitor and the second pharmaceutical composition contained in the kit may be optionally combined in the same pharmaceutical composition.

Those skilled in the art will be aware that the invention described herein is subject to variations and modifications other than those specifically described. It is to be understood that the invention described herein includes all such variations and modifications. The invention also includes all such steps, features, methods, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### EXAMPLES

### Abbreviations

+ve: positive
2-DG: [¹⁴C] 2-deoxyglucose
III: 3^{rd} ventricle
ARC: arcuate nucleus of the hypothalamus
AUC: area under curve
BAT: brown adipose tissue
chABC: Chondroitinase ABC
CPSG-ECM: chondroitin sulfate proteoglycan extracellular matrix
DAPI: 4′,6-diamidino-2-phenylindole
ddH2O: double-distilled water
EGP: endogenous glucose production
epiWAT: epididymal white adipose tissue
FITC: Fluorescein isothiocyanate
GIR: glucose infusion rate
HABP: hyaluronic acid binding protein
HFHS: high-fat high-sugar diet
HOMA-IR: Homeostatic Model Assessment for Insulin Resistance
I.C.V.: intracerebroventricular
I.N.: intranasal
I.P.: intraperitoneally
epiWAT: epididymal white adipose tissue
ingWAT: inguinal white adipose tissue
IR: insulin receptor
NPY: neuropeptide-Y
NZO: New Zealand Obese mouse
p-AKT: AKT Ser-473 phosphorylation
PF: par-fed
POMC: pro-opiomelanocortin
PV: parvalbumin
RER: Respiratory exchange ratio
RSG: Retrosplenial cortex
UCP1: uncoupling protein 1
VMH: ventromedial hypothalamus
WFA: Wisteria floribunda agglutinin

### General Procedures

### General Procedure A. Animals

Mice were maintained on a 12 h light-dark cycle in a temperature-controlled high-barrier facility with free access to food and water per NHMRC Australian Code of Practice for the Care and Use of Animals. C57BL/6J mice were sourced from the Animal Resources Centre, Australia. Animals were fed a standard chow (Barastoc, Ridley AgriProducts, Australia) or a high-fat high sugar diet (Mice: 43% and 20% of total energy from fat and carbohydrate respectively, SF04-001; Specialty Feeds, Australia. Rats 30% fats of total energy SF17-204, Specialty Feeds, Australia).

### General Procedure B. Metabolic Assessment

Metabolic measurements were undertaken in the Melbourne Mouse Metabolic Phenotyping Platform (The University of Melbourne, Australia). Glucose tolerance tests were performed on 6 h fasted conscious mice respectively by injecting D-glucose (2 mg/g of lean body mass) into the peritoneal cavity and measuring glucose in tail blood immediately before and at 15, 30, 45, 60, 90 and 120 min after injection using an Accu-Check glucometer (Roche, Germany). The areas under glucose excursion curves were determined and expressed as mmol/l×min.

Mice were acclimated for 24 h and then monitored for 48 h in an environmentally controlled Promethion Metabolic Screening System (Sable Systems International, NV) fitted with indirect open circuit calorimetry, food consumption and activity monitors to measure activity, caloric intake and energy expenditure. Fats mass was determined using mini Spec

### General Procedure C. Stereotaxic Surgery

All stereotaxic injections were undertaken under isoflurane anaesthetic using an Ultra Precise Stereotaxic Instrument (963 Kopf, Munich, Germany) or Ultra Precise Rotational Stereotaxic Instrument (69100, RWD Life Sciences, Shenzhen, China) alongside stereotaxic nanoinjectors (788130, KD Scientific, Holliston, MA) with Neurosyringes (Hamilton, NE).

To disassemble the CSPG-ECM within the ARC, mice received bilaterally (unless stated otherwise) administration of AAVs expressing U6-driven sh RNA's targeting the gene of interest or a scrambled sequence alongside a CAG driven mCherry FLEX switch. Unless otherwise stated injections were bilaterally into the ARC (coordinates, bregma: anterior-posterior, −1.70 mm; dorsal-ventral, −5.85 mm; lateral, +/−0.18 mm, 200 nl/side).

General Procedure D. Wisteria floribunda Agglutinin (WFA) Staining

CT2A cells were cultured in DMEM supplemented with 10% fetal bovine serum (FBS) and 1% penicillin-streptomycin, and passaged until passage 4 (P4), maintaining >80% confluency. Cells were then seeded at a density of 25,000 cells per well onto coverslips placed in 24-well plates. Following attachment, cells were treated with either vehicle, 4-MUX, or Odiparcil for 36 hours under standard culture conditions (37 °C, 5% CO₂). After treatment, media were immediately removed, and cells were washed then fixed with neutral buffered formalin (NBF).

Cells were incubated overnight at 4 °C with biotinylated Wisteria floribunda agglutinin (WFA; 1:1000), followed by incubation with a 647-conjugated streptavidin secondary antibody (1:1000). Nuclei were counterstained with DAPI. Coverslips were imaged using the Zeiss Axioscan 7 (using red and DAPI channels), and mean fluorescence intensity was quantified using CellProfiler.

### General Procedure E. Statistical Analysis

Statistical significance was determined by a one-way or two-way ANOVA with multiple comparisons or repeated-measures, or a one or two-tailed paired Student's t test, or ANCOVA as appropriate, or simple linear regression. p<0.05 was considered significant: * p<0.05, **p<0.01 and ***p<0.001. Statistical details of individual experiments such as exact values of n and exact statistical tests can be found in figures and legends.

### Results

To investigate the role of enzymes involved in chondroitin sulfate proteoglycan extracellular matrix (CSPG-ECM) synthesis in the development of neurofibrosis and metabolic disease, the inventors knocked down several key enzymes within the CSPG synthesis pathway in the mediobasal hypothalamus (MBH) of mice. The targeted enzymes included chondroitin sulfate N-acetylgalactosaminyltransferase 1 (CS-GalNAcT1), chondroitin sulfate N-acetylgalactosaminyltransferase 2 (CS-GalNAcT2), 4-epimerase (GALE), chondroitin sulfate synthesis 1 (CHSY1), chondroitin sulfate synthase 2 (CHPF2), chondroitin sulfate synthesis 3 (CHSY3), chondroitin polymerizing factor (CHPF), and aggrecan (ACAN).

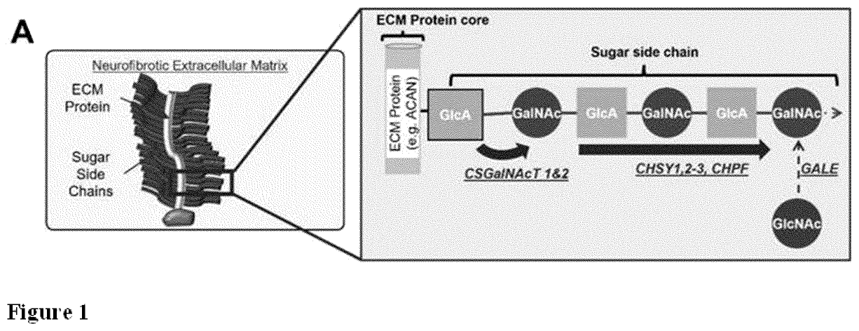

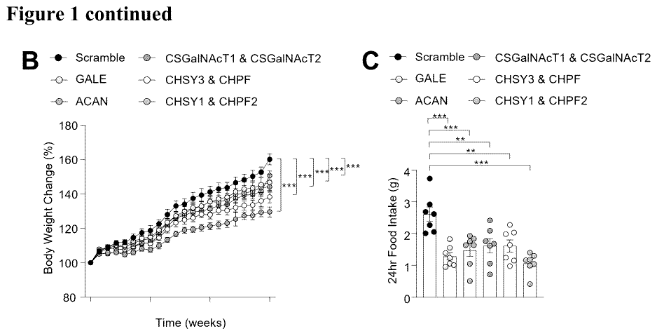

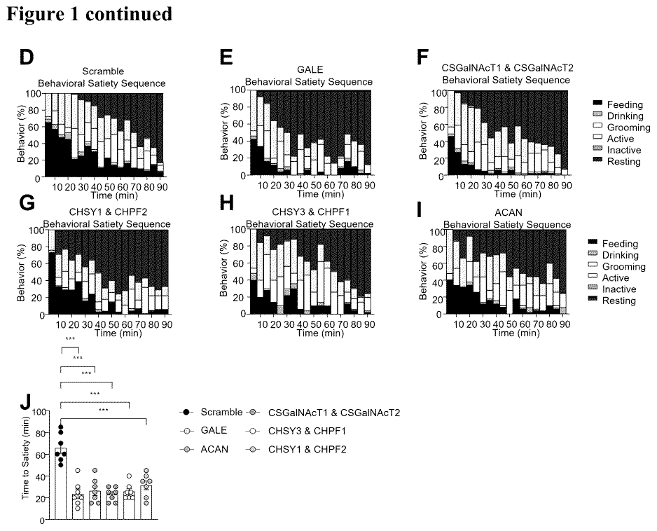

To achieve this, the inventors generated and cloned adeno-associated viruses (AAVs) encoding Cre-dependent expression of short hairpin interfering RNAs (shRNAs) targeting each of these key CSPG-ECM enzymes. These AAV-shRNAs, alongside an AAV-Cre construct, were bilaterally injected into the MBH of 8- to 10-week-old C57BL6/J mice to induce enzymatic silencing. Immediately following knockdown, mice were placed on a high-fat, high-sugar diet to induce diet-induced obesity and type 2 diabetes (FIG. 1A).

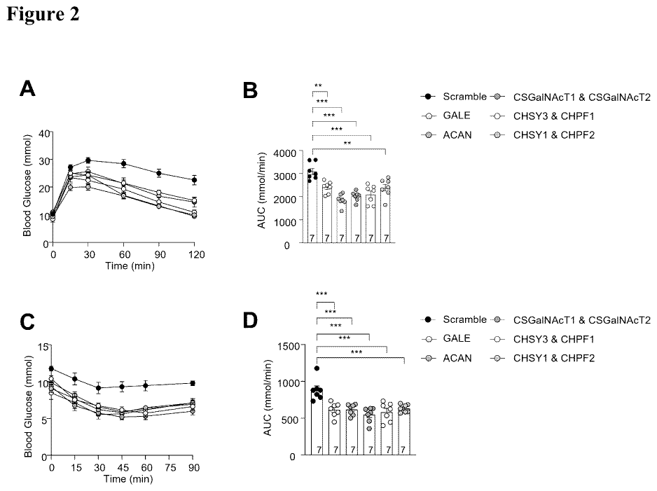

Downregulation of CSPG synthesis enzymes significantly attenuated diet-induced obesity by reducing weight gain (FIG. 1B) and suppressing appetite and food intake (FIG. 1C-J). Additionally, the knockdown of select CSPG synthases improved glycaemic control in diet-induced obesity by enhancing glucose tolerance (FIG. 2A, B) and insulin sensitivity (FIG. 2C, D).

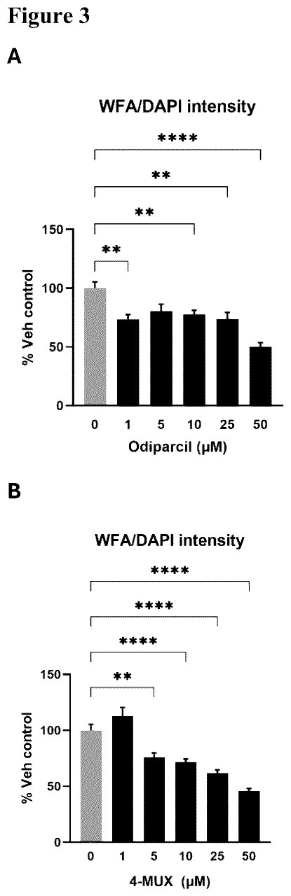

The CS-GAG inhibitors, Odiparcil and 4-MUX, inhibit in vitro CSPG deposition (FIG. 3A, B). WFA staining visualizes glycan structures rich in N-acetylgalactosamine and is primarily used to detect perineuronal nets in the nervous system. A decrease in fluorescence intensity was observed relative to the control group following the addition of Odiparcil (1, 5, 10, 25 and 50 μM) and 4-MUX (5, 10, 25 and 50 μM) meaning there was a reduction of WFA-binding CSPGs.

Taken together, these findings demonstrate the capacity of various CSPG-ECM components and synthesis enzymes as key regulators of neurofibrosis and the development of obesity, type-2 diabetes and insulin resistance.

### REFERENCES

Alonge, K. M., et al., Hypothalamic perineuronal net assembly is required for sustained diabetes remission induced by fibroblast growth factor 1 in rats. Nat Metab, 2020. 2 (10): p. 1025-1033.
Anderson, E. M., et al., Systematic analysis of CRISPR-Cas9 mismatch tolerance reveals low levels of off-target activity. J Biotechnol, 2015. 211: p. 56-65.
Chua, J S., et al., Synthetic Xylosides: Probing the Glycosaminoglycan BiosyntheticMachinery for Biomedical Applications Acc. Chem. Res. 2017. 50: p. 2693-2705.
Dodd, G. T., et al., TCPTP Regulates Insulin Signaling in AgRP Neurons to Coordinate Glucose Metabolism With Feeding. Diabetes, 2018. 67 (7): p. 1246-1257.
Dodd, G. T., et al., Intranasal Targeting of Hypothalamic PTPIB and TCPTP Reinstates Leptin and Insulin Sensitivity and Promotes Weight Loss in Obesity. Cell Rep, 2019. 28 (11): p. 2905-2922 e5.
Esko, J. D., et al., Estradiol β-D-Xyloside, an Efficient Primer for Heparan Sulfate Biosynthesis. J Biol Chem, 1991. 266 (11): p. 6674.
Keough, M. B., et al., An inhibitor of chondroitin sulfate proteoglycan synthesis promotes central nervous system remyelination. Nat Commun, 2016. 7: p. 11312.
Mirzadeh, Z., et al., Perineuronal Net Formation during the Critical Period for Neuronal Maturation in the Hypothalamic Arcuate Nucleus. Nat Metab, 2019. 1 (2): p. 212-221.
Moh, E. S. X., et al., Long-term intrathecal administration of morphine vs. baclofen: Differences in CSF glycoconjugate profiles using multiglycomics. Glycobiology, 2022. 32 (1): p. 50-59.
Olofsson, L. E., et al., Modulation of AgRP-neuronal function by SOCS3 as an initiating event in diet-induced hypothalamic leptin resistance. Proc Natl Acad Sci USA, 2013. 110 (8): p. E697-706.
Stephenson, E. L., et al. Targeting the Chondroitin Sulfate Proteoglycans: Evaluating Fluorinated Glucosamines and Xylosides in Screens Pertinent to Multiple Sclerosis. ACS Cent Sci., 2019. 5 (7): p. 1223-1234.
Tallandier M., et al. Odiparcil, a potential glycosaminoglycans clearance therapy in mucopolysaccharidosis VI-Evidence from in vitro and in vivo models. PLOS ONE 2020. 15(5): p. e0233032
Winans, K. A. and Bertozzi, C. R. An Inhibitor of the Human UDP-GlcNAc 4-Epimerase Identified from a Uridine-Based Library: A Strategy to Inhibit O-Linked Glycosylation. Chemistry & Biology, 2022. 9 (1): p. 113-129.

1. A method for treating or preventing insulin resistance or an associated disorder in a subject comprising administering an effective amount of a CS-GAG inhibitor, or pharmaceutically acceptable salt, solvate or hydrate thereof, to the subject, wherein the CS-GAG inhibitor is not a 4-epimerase inhibitor.

2. A method for treating or preventing insulin resistance or an associated disorder, supressing appetite and/or promoting weight loss in a subject, the method comprising administering to the subject an effective amount of a CS-GAG inhibitor, or pharmaceutically acceptable salt, solvate or hydrate thereof, in combination with a GLP-1 receptor (GLP-1R) agonist, or pharmaceutically acceptable salt or solvate thereof, wherein the CS-GAG inhibitor is not a 4-epimerase inhibitor.

3. A pharmaceutical combination comprising (i) a CS-GAG inhibitor, or pharmaceutically acceptable salt, solvate or hydrate thereof, and (ii) a GLP-1 receptor (GLP-1R) agonist, or pharmaceutically acceptable salt, solvate or hydrate thereof, wherein the CS-GAG inhibitor is not a 4-epimerase inhibitor.

4. The method of claim 1, wherein the associated disorder is selected from prediabetes, type-2 diabetes mellitus, obesity, metabolic syndrome, hypertension, dyslipidemia, atherosclerosis, non-alcoholic fatty liver disease (NAFLD), polycystic ovary syndrome (PCOS), coagulopathy, insulin resistance syndrome, hyperglycemia, retinopathy, neuropathy, nephropathy, macrovascular disease, and combinations thereof.

5. The method of claim 4, wherein the associated disorder is a metabolic disease.

6. The method of claim 5, wherein the metabolic disease is type-2 diabetes mellitus.

7. The method of claim 5, wherein the metabolic disease is characterized by insulin resistance.

8. The method of claim 4, wherein the associated disorder is obesity.

9. The method of claim 1, wherein the CS-GAG inhibitor is selected from the group consisting of:
xylosyltransferase 1 inhibitor,
xylosyltransferase 2 inhibitor,
galactosyltransferase 1 inhibitor,
galactosyltransferase 2 inhibitor,
glucuronyltransferase 1 inhibitor
chondroitin sulfate N-acetylgalactosaminyltransferase 1 inhibitor,
chondroitin sulfate N-acetylgalactosaminyltransferase 2 inhibitor,
chondroitin sulfate synthase 1 inhibitor,
chondroitin sulfate synthase 2 inhibitor,
chondroitin sulfate synthase 3 inhibitor,
chondroitin polymerizing factor 2 inhibitor,
glycosaminoglycan xylosylkinase (FAM20B) inhibitor,
2-phosphoxylose phosphatase 1 (PXYLP1) inhibitor,
chondroitin 4-O-sulfotransferase 1 inhibitor,
carbohydrate sulfotransferase 15 (GalNac4S-6ST) inhibitor,
carbohydrate sulfotransferase 8 (CHST8) inhibitor,
chondroitin-6-O-sulfotransferase (CS6ST1-2) inhibitor,
an inhibitor of versican expression or synthesis,
an inhibitor of phosphocan expression or synthesis,
an inhibitor of neurocan expression or synthesis,
an inhibitor of brevican expression or synthesis,
an inhibitor of aggrecan expression or synthesis,
a pharmaceutically acceptable salt, solvate or hydrate thereof, and
combinations thereof.

10. The method of claim 1, wherein the CS-GAG inhibitor is selected from the group consisting of:
galactosyltransferase 1 inhibitor,
chondroitin sulfate N-acetylgalactosaminyltransferase 1 inhibitor,
chondroitin sulfate synthase 1 inhibitor,
chondroitin sulfate synthase 3 inhibitor,
an inhibitor of aggrecan expression or synthesis,
a pharmaceutically acceptable salt, solvate or hydrate thereof, and
combinations thereof.

11. The method of claim 1, wherein the CS-GAG inhibitor is selected from the group consisting of:
β-D-xyloside,
β-D-thioxyloside,
a pharmaceutically acceptable salt, solvate or hydrate thereof, and
combinations thereof.

12. The method of claim 1, wherein the CS-GAG inhibitor is selected from the group consisting of
Odiparcil,
methylumbelliferone β-D-xylopyranoside,
a pharmaceutically acceptable salt, solvate or hydrate thereof, and
combinations thereof.

13. The method of claim 2, wherein the GLP-1R agonist is GLP-1 or an analogue thereof.

14. The method of claim 2, wherein the GLP-1R agonist is selected from liraglutide dulaglutide, exenatide, semaglutide and lixisenatide, or any combination thereof.

15. The method of claim 14, wherein the GLP-1R agonist is liraglutide.

16. The method of claim 2, wherein the GLP-1R agonist is a GLP-IR co-agonist.

17. The method of claim 16, wherein the GLP-1R co-agonist is tirzepatide or LY3437943.

18. The method of claim 1, wherein the CS-GAG inhibitor is administered orally.

19. The method of claim 2, wherein the GLP-1R agonist is administered by injection or orally.

20. The method of claim 1, wherein the subject exhibits one or more risk factors selected from:
obesity,
body mass index greater than or equal to 25 kg/m², 30 kg/m², 35 kg/m² or 40 kg/m²,
hyperglycemia,
fasting blood glucose concentration of greater than or equal to 125 mg/dL, 135 mg/dL, 140 mg/dL, 145 mg/dL, 150 mg/dL, 155 mg/dL or 160 mg/dL,
a fasting high triglyceride (TG) concentration of between about 150 mg/dl and about 500 mg/dL, about 150 mg/dL and about 200 mg/dL, about 200 mg/dL and about 400 mg/dL, about 400 mg/dL and about 500 mg/dL,
hemoglobin A1c levels greater than or equal to 5.7%, 6.0%, 6.5%, 7.0%, 7.5%, 8.0% or 8.5%
a fasting plasma/serum insulin assay greater than 15 mIU/L, 20 mIU/L, 25 mIU/L,
a LDL-c level greater than 100 mg/dL
a HDL-c level less than 60 mg/dL and
combinations thereof.
